(19) United States
(12) Patent Application Publication
(10) Pub. No.: US 2005/0113366 A1
(43) Pub. Date: May 26, 2005

(54) Derivatives of 4-hydroxybutanoic acid and of its higher homologue as ligands of $g(g)-hydroxybutyrate (ghb) receptors, pharmaceutical compositions containing same and pharmaceutical uses

(76) Inventors: Jean-Jacques Bourguignon, Illkirch (FR); Michel Maitre, Strasbourg (FR); Evelyne Klotz, Mutzig (FR); Martine Schmitt, Strasbourg (FR); Serge Gobaille, Vendenheim (FR); Jean-Paul Macher, Bergholtz-Zell (FR)

Correspondence Address: David L Parker;Fulbright & Jaworski, Suite 2400, 600 Congress Avenue, Austin, TX, 78701, US

(21) Appl. No.: 10/432,692

(22) PCT Filed: Nov. 16, 2001
(86) PCT No.: PCT/FR01/03615

(30) Foreign Application Priority Data
Nov. 27, 2000 (FR) 00/15291

(51) Int. Cl. A61K 31/55; A61K 31/5513; A61K 31/503; A61K 31/53

(52) U.S. Cl. 514/221; 514/243; 514/248; 514/303; 514/383; 514/411

(57) ABSTRACT

The invention concerns the field of synthesis organic chemistry applied to the pharmaceutical field and concerns novel derivatives of 4-hydroxybutanoic acid and its higher homologue, 5-hydroxypentanoic acid, their crotonic homologues, pharmaceutical compositions containing them and their pharmaceutical uses. Said novel derivatives are capable of binding with γ-hydroxybutyrate (GHB)-specific receptors and hence capable of exhibiting agonist or antagonist properties, in particular for treating sleep disorders, anxiety and general diseases of the central nervous system. The invention also concerns compounds of general formula (I) wherein the substituents are as defined in the description.

The present invention relates to the field of synthesis organic chemistry applied to the pharmaceutical field and relates to new derivatives of 5-hydroxybutanoic acid and its higher homologue, 5-hydroxypentanoic acid, of their crotonoid homologues, to the pharmaceutical compositions containing them and to their pharmaceutical uses. These new derivatives are capable of binding to the specific receptors of γ-hydroxybutyrate (GHB) and are consequently capable of having agonist and antagonist properties.

The present invention also relates to bioprecursors, isosteres and prodrugs of these compounds with improved oral therapeutic efficacy. Finally, the present invention relates also to the specific uses of these GHB mimetics, particularly in the treatment of sleep disorders, anxiety and general disorders of the central nervous system (CNS).

γ-hydroxybutyrate was studied as early as the 1960s by Laborit, as an isosteric analogue of γ-aminobutyric acid (GABA) which is capable of passing through the blood-brain barrier. In fact, GHB has been identified as being a natural component of the mammalian brain and is currently used in therapy as a well-tolerated general anesthetic.

Its catabolism is very rapid, the half-life being, as an indication, approximately one hour in cats. GHB induces deep sedation with loss of postural reflexes and analgesia. The electroencephalogram has been interpreted sometimes in certain animal species as being a sleep graph without paradoxical sleep disturbance, and sometimes as being a graph reminiscent of that of a “petit mal” epileptic episode. The latter problem reduced the indications of GHB in anesthesia in elderly patients.

It was possible to demonstrate in the 1980s that GHB fulfilled the criteria generally used for qualification of a substance as neurotransmitter or neuromodulator, namely the presence of the synthetic enzyme of this compound in the presynaptic ends, calcium-dependent release by membrane depolarization, sodium-dependent high affinity active transport and, finally, high affinity saturable reversible binding on membrane preparations of synaptic origin.

All these properties make it possible to establish the existence of neuronal circuits in which a role of γ-hydroxybutyrate is directly implied.

The present invention aims to propose new derivatives of 5-hydroxybutanoic acid, 5-hydroxypentanoic acid and of their crotonoid homologues, which are capable of binding more effectively to the specific receptors of γ-hydroxybutyrate (GHB) and which have agonist and antagonist properties.

The inventors unexpectedly revealed that by functionalizing compounds of the family of 4-hydroxybutanoic acid or of 5-hydroxypentanoic acid (the acid, its esters, its sodium salts, its crotonoid derivatives, etc.), in particular by aromatic substitution of the methyl carbon at the end of the chain, it was possible to obtain synthetic derivatives with increased affinity for GHB receptors compared to the affinity of natural GHB for these same receptors.

In a first aspect, the present invention relates to the compounds with general formula I: 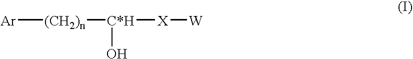
in which Ar represents one of the following mono-, bi- or tricyclics: 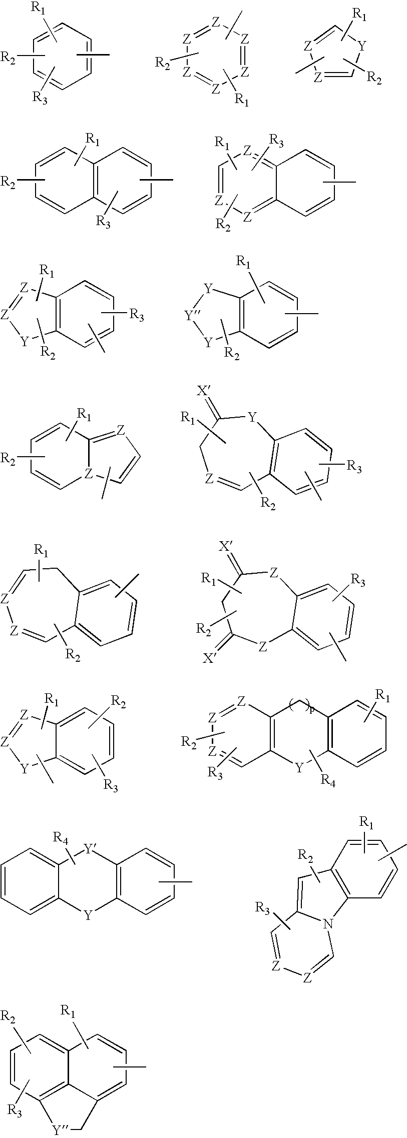
in which:
R₁, R₂, R₃ and R₄ independently represent a hydrogen atom, a halogen, an alkyl group, an aryl group, an aralkyl group, a hydroxyl group, a methoxy group, an acetyl group, a tosyl group, a COOEt group, an NHCOCH₃ group, an NH₂ group, a CON(CH₃)₂ group, an NO₂ group or a COR₅R₆ group, in which R₅ and R₆ independently represent a hydrogen atom, a methyl group, a CH₃H₇ group or a benzyl group,
each z independently represents a nitrogen or carbon atom,
Y and Y′ independently represent a carbon, sulfur, oxygen or nitrogen atom,
Y″ represents a methylene, ethylene or propylene group,
each X′ independently represents a sulfur or oxygen atom,
p has a value of 0, 1 or 2,
in which n has a value of 0 or 1,
in which X independently represents (CH₂)₂ or (CH₂)₃ or X═—CH═CH— (trans) and in which W represents COOH, COO⁻M⁺ (M⁺ representing a counterion which is pharmaceutically acceptable), CH₂OH, COOR (with R representing an alkyl group), SO₃H or PO₃H₂ or a group chosen from the following: 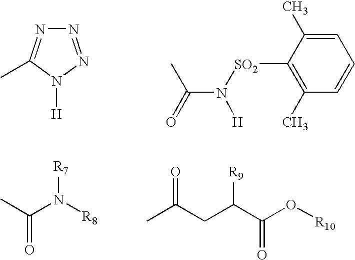
   in which R₇ and R₈ independently represent a hydrogen atom, an alkyl group, an aryl group, an aralkyl group or a hydroxyl group,
in which R₉ independently represents a hydrogen atom or a methyl group and in which R₁₀ independently represents an ethyl, C₁₂H₁₅ or adamantyl group.

More preferably, it relates to the compounds with general formula I′: 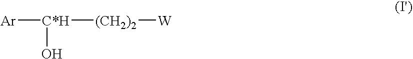
in which W represents COOH or COO⁻M⁺ (M⁺ representing a counterion which is pharmaceutically acceptable), and in which Ar represents the groups defined above.

In particular, it relates to the salts with general formula I″ 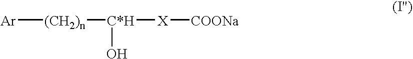
in which Ar, X and n are defined above, which are obtained from compounds with general formula I and which can be prepared with techniques known to the expert in the field.

In a second aspect, the present invention provides pharmaceutical compositions which contain, as active ingredient, at least one compound with general formula I, I′ or I″. It also relates to the use of the new synthesized derivatives for therapeutic purposes.

The present invention describes different routes of synthesis, relating to preferred embodiments, which are given as nonlimiting examples, and which are explained with reference to the appended drawings in which:

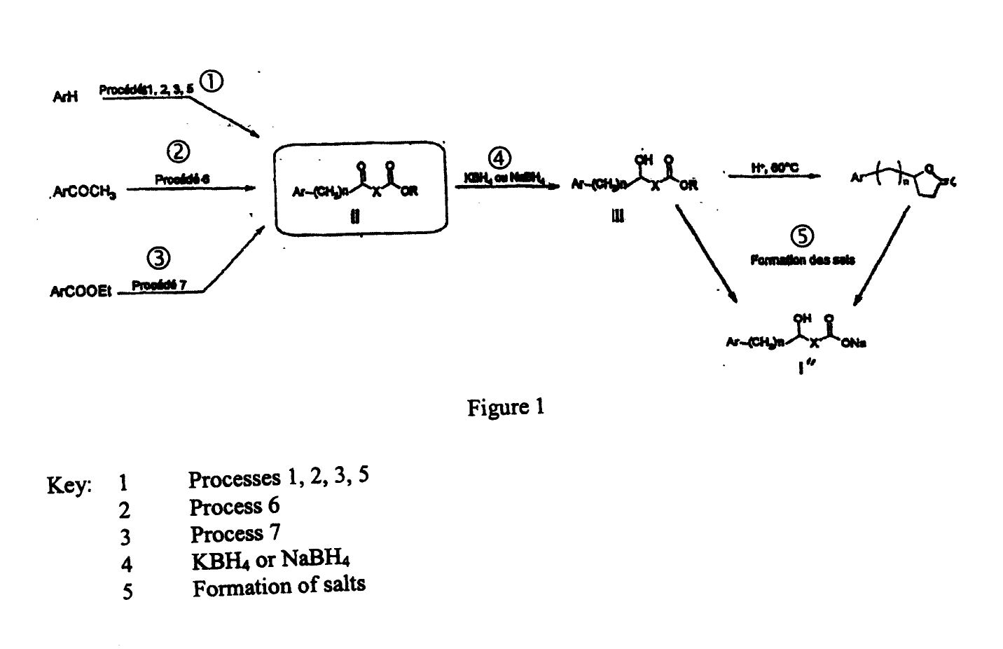

FIG. 1 represents a simplified block diagram of the different processes of synthesis of the compounds according to the invention as well as of their main intermediate reaction products,

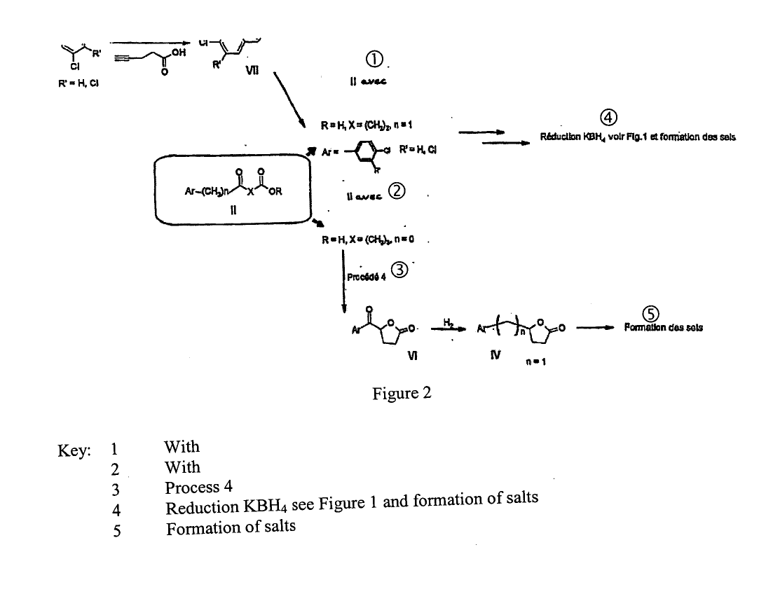

FIG. 2 represents a simplified diagram of other processes of synthesis of compounds according to the invention,

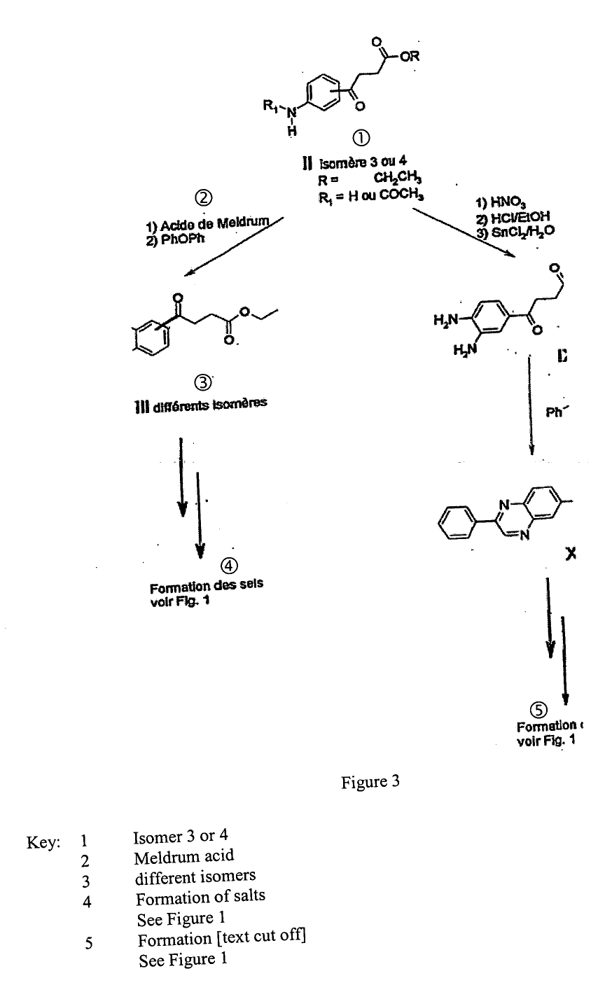

FIG. 3 represents another simplified diagram of other processes of synthesis of compounds according to the invention; and

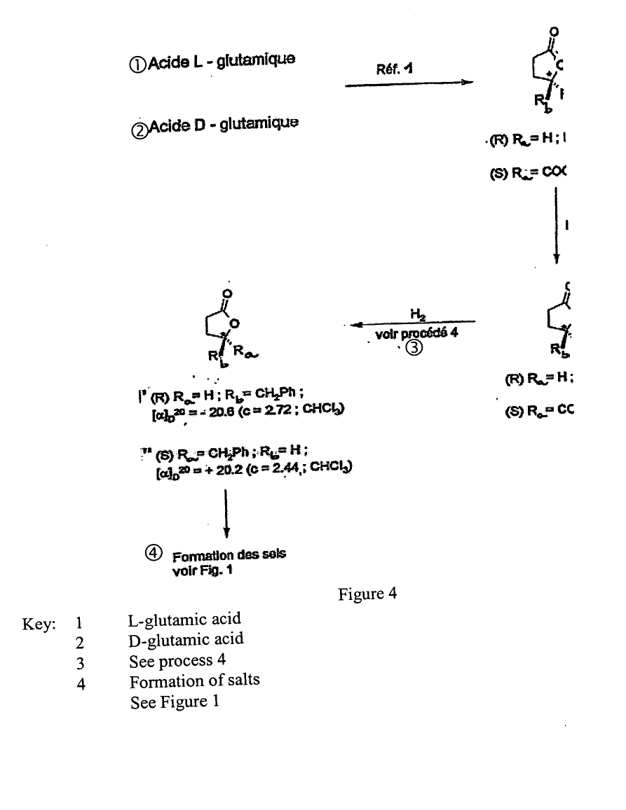

FIG. 4 represents a simplified block diagram of a process of synthesis of enantiomerically pure compounds according to the invention as well as of their main intermediate reaction products.

Except when indicated otherwise, the following terms used in the present description and the present set of claims have the following meanings:
“alkyl” designates a straight saturated monovalent hydrocarbon radical with 1 to 6 carbon atoms or a branched saturated monovalent hydrocarbon radical with 3 to 6 carbon atoms, for example, methyl, ethyl, propyl, 2-propyl, butyl, etc.
“aryl” designates an aromatic hydrocarbon radical of the phenyl type, possibly substituted, that is, a phenyl group which is possibly substituted independently with one or two substituents chosen from the group formed by the halogen, —OR (in which R represents a hydrogen atom or an alkyl group) and —NRR′ (in which R and R′ independently represent a hydrogen atom or an alkyl group) groups.
“aralkyl” designates an aryl radical as defined above substituted by an alkylene group, that is, a straight saturated bivalent hydrocarbon radical with 1 to 6 carbon atoms or a branched saturated bivalent hydrocarbon radical with 3 to 6 carbon atoms containing at least one double bond, such as benzyl, phenylethyl, etc.
“halogen” designates a monovalent radical chosen from fluorine, chlorine, bromine and iodine.
The expressions “carbon atom” and “nitrogen atom” must be understood to designate said atoms, possibly provided with one or two hydrogen atoms and/or substituted by the aforementioned groups and/or having a chemical bond, in such a way as to satisfy the expected valences of carbon and nitrogen. As examples, z can represent, depending on the case, N, NH, C, CH or CH₂, Y can be, depending on the case, C, CH, CH₂, S, O, etc.

The compounds according to the present invention can have one or more asymmetrical centers and can therefore be produced as individual stereoisomers (R) or (S), or as mixtures of them. Except when specifically indicated, the present description and the present set of claims include both the individual enantiomers and their mixtures in any proportions, including the so-called racemic mixtures.

“Pharmaceutically acceptable excipient” designates a single excipient or a set of excipients which can be used in the preparation of a pharmaceutical composition which is generally safe, nontoxic and not undesirable biologically or otherwise, and includes one or more excipients which are acceptable for veterinary as well as for human pharmaceutical use.

“Pharmaceutically acceptable salt” designates a salt or a mixture of several salts which can be used in the preparation of a pharmaceutical composition which is generally safe, nontoxic and not undesirable biologically or otherwise and which has the desired pharmacological activity of the parent molecule. Such salts include the salts formed when an acid proton of the parent molecule is replaced by a metal ion, for example, an alkali metal ion (preferably sodium or lithium) or an alkaline-earth ion, or is coordinated with an organic base such as ethanolamine, diethanolamine, the amino acids (lysine in particular), etc.

A “pharmaceutically acceptable counterion” designates an ion with a charge opposite that of the substance with which it is associated and which is pharmaceutically acceptable.

The term “prodrug” designates any compound which releases an active related drug according to general formula I, I′ or I″ in vivo when such a prodrug is administered to a mammalian subject. The prodrugs are prepared by modifying the functional groups which are present in such a way that the modifications can be eliminated or neutralized in vivo in order to release the parent molecule, examples of prodrugs including, in a nonlimiting manner, esters (for example, the derivatives of acetate, formate, benzoate, etc.), etc.

The terms “treating” or “treatment” of a disease include:
preventing the disease, namely, causing the clinical symptoms of the disease not to develop in a mammal who may be exposed or predisposed to the disease but who is not experiencing or who does not yet have symptoms of the disease,
inhibiting the disease, namely, stopping or reducing the development of the disease or of its clinical symptoms, or
causing the disease to disappear, namely, bringing about the regression of the disease or of its clinical symptoms.

A “therapeutically effective quantity” designates the quantity of a compound which, when administered to a mammal for treating a disease, is sufficient to bring about such a treatment for the disease. This quantity will vary as a function of the compound, the disease and its severity, and the age, weight, etc., of the mammal who is to be treated.

The starting compounds can be obtained commercially or can be synthesized according to the conventional processes. It is understood that the present application is not limited to a particular route of synthesis and extends to other processes making production of the indicated compounds possible.

The compounds with general formula I″ are prepared as illustrated in the diagram of FIG. 1. The key intermediate products are the acids with general formula I and the corresponding keto acids or esters with general formula II:
Ar—(CH₂)ₙ—(C*═O)—X—CO₂R (II)
in which R can be hydrogen or an alkyl group, preferably ethyl or methyl.

Depending on the nature of the aromatic (Ar), these derivatives are obtained by the Friedel-Crafts reaction (M. Kakushima et al. JOC, 48(19), 1983, 3214; G. J. Quallich et al. JOC, 55(16), 1990, 4971) or by condensation of a methylketone with glycoxylic acid (J. J. Bourguignon et al. J. Med. Chem., 1988, 31, 893-897) or by reaction of ArCO₂Et with diethyl succinate in a basic medium (H. Michel et al. Arch. Pharmaz., 1974, 307, 689). Application examples will be given in part A) hereafter (cf., in particular, Table 1 of this part). These keto acids or esters II are subsequently reduced to corresponding alcohols with general formula III, using NaBH₄ in the case in which R=Me or R=Et, and using KBH₄ if R═H, and then salified using NaOH in order to obtain the corresponding sodium salts with general formula I″, as represented in FIG. 1.

As indicated also in the diagram of FIG. 1, the reduced compounds III are lactonized into compounds with general formula IV by simple heating in an acid medium. This reaction is well-known to the expert in the field and does not need to be developed in more detail here. Of course, the expert in the field will be able to choose any acid suitable for obtaining the desired lactones. As an example of a particularly suitable acid, it is possible to mention hydrochloric acid, as illustrated in a nonlimiting manner in the examples which follow. The reaction of an amine with the lactones IV leads to the corresponding amides with general formula V.

Furthermore, the keto acids for which R═H, X═(CH₂)₃ and n=0 can be transformed into γ-aroyl-γ-butyrolactones with general formula VI by reaction with bromine (K. Yamada et al., Tetrahedron, 1971, 27, 5445-5451). These lactones VI can then be reduced to γ-benzyllactones with general formula IV by catalytic hydrogenation. However, this last process cannot be applied if the aromatic Ar carries a halogen. In this case, a reaction catalyzed by Pd(OAc)₄ is called for. Thus, by treating, for example, 1-chloro-4-iodobenzene, the methylidine-γ-butyrolactone with general formula VII is obtained (A. Arcadi et al. JOC, 1992, 57, 976-982) which, by action of NaOH, leads to the corresponding keto acid II (cf. FIG. 2).

The different steps mentioned in the preceding for synthesis of the compounds according to the invention will now be described in more detail by means of nonlimiting illustrative examples.

### A) Access to the Intermediate Keto Acids or Keto Esters (II)

The formation of the intermediate keto acids or keto esters with general formula II is described hereafter, the different processes for obtaining them being summarized in Table 1 which follows.

TABLE 1
Processes for obtaining intermediate keto acids or keto esters

| | | | | | ^{\mathring{2}}Procédé |
|---|---|---|---|---|---|
| ^{\mathring{1}}Réactifs de départ | | X | n | R | n° |
| ArH | 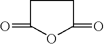 | (CH₂)₂ | 0 | H | 1 |
| ArH | 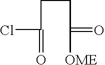 | (CH₂)₂ | 0 | Me | 2 |
| ArH | 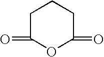 | (CH₂)₃ | 0 | H | 3 |
| ArH | 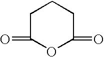 | CH═CH | 1 | H | 4 |
| ArH | 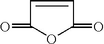 | CH═CH | 0 | H | 5 |
| ArCOCH₃ | 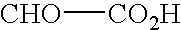 | (CH₂)₂ | 0 | H | 6 |
| ArCO₂Et | 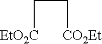 | (CH₂)₂ | 0 | H | 7 |
| 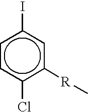 | 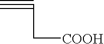 | (CH₂)₂ | 1 | H | 8 |

Key:
¹Starting reagents
²Process No.

Eight processes and eleven examples of embodiments will be detailed in the following. The keto esters which are obtained are reduced to lactones and salified as indicated in the diagram of FIG. 1.

### 1) Process 1: Friedel-Crafts Reaction with Succinic Anhydride

Four examples specifying the conditions of operation of solvents, temperature and reaction time are described hereafter.

### Example 1

### Synthesis of 4-[5-ethoxycarbonyl)-1H-pyrrol-3-yl]-4-oxobutanoic acid (keto acid derivative of Compound No. 9)

Succinic anhydride (2.80 g, 27.9 mmol) is dissolved in dichloro-1,2-ethane (20 mL). Aluminum chloride (11.20 g, 83.9 mmol) is added and then the pyrrolic derivative (2.00 g, 14.4 mmol) dissolved in the solvent (20 mL). The reaction medium is stirred at room temperature for 3 h and then poured over ice. The precipitate which forms is filtered and dried.

| | |
|---|---|
| beige solid MM(C₁₁H₁₃NO₅): 239.23 g mass: 2.62 g Yield: 76% | 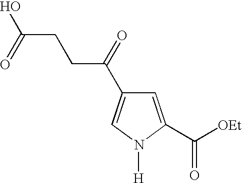 |

^{\mathring{1}}RMN(¹H, 300 MHz, DMSO): 12, 51^{\mathring{2}}(large s, 1H, COOH); 7, 75(s, 1H, CHpyrrol.); 7, 15(s, 1H, CHpyrrol); 4, 27(q, 2H, —CH₂—, J³ = 7, 0 Hz); 3, 04(t, 2H, —CH₂—, J³ = 6, 2 Hz); 2, 52(t, 2H, —CH₂—, J³ = 6, 2 Hz); 1, 30(t, 3H, —CH₃, J³ = 7, 0 Hz).
Key:
¹NMR*
²broad
*[Editor's note: Within numbers in the NMR data and in the tables which follow, commas represent decimal points.]

### Example 2

### Synthesis of 4-(2,3-dihydro-1H-inden-5-yl)-4-oxobutanoic acid (keto acid derivative of Compound No. 15)

A conventional Friedel-Crafts reaction is carried out in nitrobenzene (60 mL) at room temperature for 4 h: indane (4 mL, 32.66 mmol), AlCl₃ (11.00 g, 82.5 mmol) and succinic anhydride (3.00 g, 30 mmol). A slightly yellow solid is obtained, which is characterized as follows:

| | |
|---|---|
| slightly yellow solid MM (C₁₃H₁₄O₃): 218.25 g mass: 5.03 g Yield: 71% | 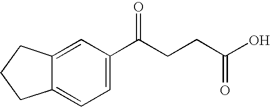 |

^{\mathring{1}}RMN(¹H, 200 MHz, DMSO): 12, 13(large s, 1H, —COOH); 7, 84(s, 1H, CHarom); 7, 78(d, 1H, CHarom., J³ = 7, 8 Hz); 7, 37(d, 1H, CHarom., J³ = 7, 8 Hz); 3, 23(t, 2H, —CH₂—, J³ = 5, 9 Hz); 2, 93(m, 4H, 2- CH₂—); 2, 57(t, 2H, —CH₂—, J³ = 5, 9 Hz); 2, 05(m, 2H, —CH₂—).
Key:
¹NMR
²Broad

### Example 3

### Synthesis of 4-(5-acetyl-10,11-dihydro-5H-dibenzo[b,f]azepin-3-yl)-4-oxobutanoic acid (keto acid derivative of Compound No.30)

The N-acetylated derivative of azepine (1.00 g, 4.21 mmol) is dissolved in CS₂ (10 mL), and aluminum chloride (5.62 g, 42.1 mmol) is added. The succinic anhydride (3.37 g, 33.8 mmol) is then added in small spatula loads. The reaction medium is stirred (mechanical stirring) at reflux for 6 h. After cooling, this solution is poured over ice and extracted with ethyl acetate. The organic phase is treated with 1N NaOH, and the aqueous phase is washed with ethyl acetate. Then, it is then treated again in acid medium by addition of concentrated HCl. The product is extracted with ethyl acetate, and dried using Na₂SO₄, and the solvents are evaporated.

The oil which is obtained is purified by silica gel column chromatography with a mixture of CH₂Cl₂/MeOH 9/1 as eluent.

| | |
|---|---|
| yellow oil MM(C₂₀H₁₉NO₄): 337.38 g mass: 900 mg Yield: 63% | 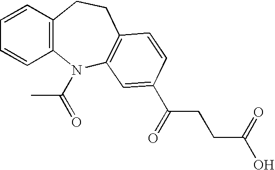 |

^{\mathring{1}}RMN(¹H, 300 MHz, DMSO): 12, 17^{\mathring{2}}(large s, 1H, COOH); 8, 11-7, 22(m 7H, 7CHarom.); 3, 47-3, 22(m, 4H, —CH₂—CH₂—); 2, 94-2, 84(m, 2H, —CH₂—); 2, 59(t, 2H, —CH₂—, J³ = 6, 2 Hz);1, 93 (s, 3H, —CH₃).
Key:
¹NMR
²Broad

### Example 4

### Synthesis of 4-[4-(acetylamino)phenyl]-4-oxobutanoic acid (keto acid derivative of Compound No. 8)

With mechanical stirring and argon flow, at room temperature, DMF (22 mL) is added dropwise to aluminum chloride (134 g, 1.00 mol). After complete addition, the temperature is brought to 90° C., and the acetanilide (10 g, 74 mmol) and succinic acid (10 g, 100 mmol) mixture is added in small spatula loads. The reaction medium is stirred at 80-90° C. for 2 h, and then poured over 500 g of crushed ice. By addition of concentrated HCl, the pH is brought to 1. The precipitate which forms is filtered and recrystallized in DMF/H₂O.

| | |
|---|---|
| pale-yellow powder MM(C₁₂H₁₃NO₄): 235.24 g mass: 12.10 g Yield: 70% | 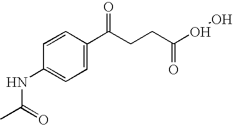 |

^{\mathring{1}}RMN(¹H, 300 MHz, DMSO): 12, 13^{\mathring{2}}(large s, 1H, COOH); 10, 31(s, 1H, NH); 7, 95(d, 2H, 2CHarom., J³ = 8, 7 Hz); 7, 74(d, 2H, 2CHarom., J³ = 8, 7 Hz); 3, 21(t, 2H, —CH₂—, J³ = 6, 2 Hz); 2, 58(t, 2H, —CH₂—, J³ = 6, 2 Hz); 2, 11(s, 3H, —CH₃).
Key:
¹NMR
²Broad

### 2) Process 2: Friedel-Crafts reaction with methyl-4-chloro-4-oxobutanoate

### EXAMPLE

### Synthesis of methyl 4-(7-methylimidazo[1,2-a]pyridin-3-yl)-4-oxobutanoate (methyl keto ester derivative of Compound No. 21)

7-methylimidazo[1,2-a]pyridine (0.50 g, 3.62 mmol) is dissolved in CS₂ (8 mL). The solution is cooled to 0° C., and aluminum chloride (1.40 g, 5.35 mmol) is added in small portions. It is stirred for 30 min, and then the reaction medium is brought to reflux. An acid chloride (1.50 g, 10 mmol) is added, and it is stirred at reflux for 2 h. The solution is cooled and poured over ice. The pH is adjusted to 9 by addition of 3N NaOH. It is extracted with ethyl acetate and then filtered using diatomaceous earth. The ethyl acetate phase is dried using sodium sulfate and then evaporated to dryness. Finally, it is triturated with ether, and the product obtained is filtered.

| | |
|---|---|
| light-maroon powder MM(C₁₃H₁₄N₂O₃): 246.26 g mass: 100 g Yield: 12% | 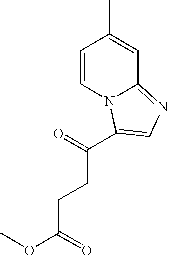 |

^{\mathring{1}}RMN(¹, 300 MHz, CDCl₃): 9, 50(d, 1H, CHarom., J³ =6, 7 Hz); 8, 40(s, 1H, CHimidaz.); 7, 50(s, 1H, CHarom.); 6, 91(d, 1H, CHarom., J³ = 6, 7 Hz); 3, 75(s, 3H, —CH₃); 3, 28(t, 2H, —CH₂—, J³ =6, 7 Hz); 2, 81(t, 2H, —CH₂—, J³ = 6, 7 Hz); 2, 50 (s, 3H, —CH₃).
Key:
1 NMR

### 3) Process 3: Friedel-Crafts reaction with glutaric anhydride

### EXAMPLE

### Synthesis of 4-(4-methoxybenzoyl)butyric acid

In a dry 250-mL two-necked flask in an argon atmosphere at 0° C., glutaric acid (5 g, 87.64 mmol) is added to a mixture of CH₂Cl₂ (125 mL) and anisole (14.2 g, 1.5 Eq., 0.13 mol). AlCl₃ (12.3 g, 1.05 Eq, 92 mmol) is added in portions, and the mixture is stirred at 0° C. for 4 h before being poured over ice (100 g). The precipitate is collected by filtration, washed with cold water and dried under vacuum. 9 g of the desired product are obtained.

| | |
|---|---|
| Yield: 50% White solid, MP = 133° C. | 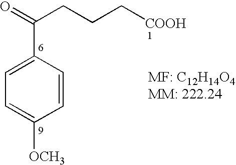 |

^{\mathring{1}}RMN¹H(200 MHz, CDCl₃): δ 2, 08(p, 2H, ³J = 7, 2 Hz, H3); 2, 50(t, 2H, ³J = 7, 2 Hz, H2); 3, 04(t, 2H, ³J = 7, 2 Hz, H4); 3, 88(s, 3H, OCH₃); 7, 47(syst{acute over (e+0 me AB, 4H arom, J_{AB} +L =+0 8, 8 Hz, Δν+0 =+0 203 Hz))}
Key:
¹NMR
²System
^{\mathring{1}}RMN ¹³C(50 MHz, CDCl₃): δ 19, 18(C3); 33, 04 ²(C2 ou C4); 36, 94(C2 ou C4); 55, 46(OCH₃); 113, 72(C8); 129, 78(C6); 130, 30(C7); 163, 48(C9); 178, 80(Cl); 197, 99(C5).
Key:
¹NMR
²Or

| Microanalysis: | C % | H % |
|---|---|---|
| theoretical | 64.85 | 6.35 |
| actual | 64.71 | 6.20 |

^{\mathring{1}}SM(IE, 70 eV) m/e(intensité relative %): 222(M⁺, 78), 135 [[(M-(CH₂)₃COOH)⁺, 100].
Key:
¹Mass spectrometry
²Relative intensity %

### 4) Process 4: Friedel-Crafts Reaction with Glutaric Anhydride Followed by the Action of Bromine

This process consists of a direct switch to 5-benzyllactone. First of all, a Friedel-Crafts reaction is performed between the chosen aromatic nucleus and glutaric anhydride (cf. Process 3), followed by a substitution with bromine and then lactonization in basic medium.

### EXAMPLE

### Synthesis of 5-benzoyldihydrofuran-2-one

In a 500-mL round-bottomed flask, 4-benzoylbutyric acid (20 g, 0.1 mol) is added to a mixture of dioxane (180 mL) and ether (60 mL). Bromine (6.55 mL, 1.25 Eq, 0.125 mol) is added dropwise, and the solution is stirred for 3 h and then poured into an aqueous 30% NaHCO₃ solution (150 mL) and again stirred for 6 h. The phases are separated, and the aqueous phase is extracted with CH₂Cl₂ (3×75 mL). The organic phases are combined, washed with brine (150 mL), dried using anhydrous sodium sulfate, filtered and evaporated to dryness. Silica column chromatography gives the desired compound (14.64 g).

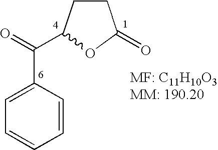

Chromatography solvent: AcOEt/hexane, 30/70, R_{f}: 0.20 Yield: 74% White solid, MP = 91° C.
^{\acute{1}}RMN ¹H(200 MHZ, CDCl₃): δ 2, 44-2, 67(m, 4H, H2 et H3); 5, 79-5, 85(m, 1H, H4); 7, 48-7, 65(m, 3H arom. meta et para); 7, 96-8, 00(m, 2H arom. ortho).
^{\acute{1}}RMN ¹³C(50 MHz, CDCl₃): δ 24, 92(C3); 26, 76(C2); 78, 18(C4); 128, 75(CH arom.); 128, 97(CH arom.); 133, 49(C6); 134, 29 (CH arom.); 176, 20(C1); 194, 24(C5).
Key:
¹NMR
²Meta and para

| Microanalysis: | C % | H % |
|---|---|---|
| theoretical | 69.46 | 5.30 |
| actual | 69.36 | 5.29 |

^{\acute{1}}SM(IE, 70 eV) m/e(intensité relative %): 190(M⁺, 38), 105[(M-PhCO)⁺, 100].
Key:
¹Mass spectrometry
²Relative intensity %

The keto lactones with general formula VI which are obtained are then reduced by H₂ using Pd/C to give the compounds with general formula VI and the corresponding salts (cf. FIG. 2).

### EXAMPLE

### Synthesis of 5-benzyldihydrofuran-2-one

To a solution of the keto lactone synthesized in the preceding (1 g, 5.25 mmol) in methanol (20 mL), Pd/C 10% (10 wt%, 0.1 g) and concentrated HCl (0.3 mL) are added. The mixture is hydrogenated in a Paar apparatus at 70 psi (483 kPa) for 16 h and then filtered using Celite, washing with ethyl acetate. Evaporation of the solvents and silica column chromatography give the desired compound (556 mg).

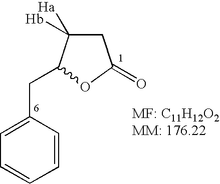

Chromatography solvent: AcOEt/hexane, 20/80, R_{f}: 0.23 Yield: 60% Colorless oil

^{\mathring{1}}RMN ¹H(200 MHz, CDCl₃): δ 1, 85-1, 97 (m, 1H, Ha ou²Hb); 2, 13-2, 48(m, 3H, H2^{\acute{3}}et Ha^{\acute{2}}ou Hb); 2, 84-3, 08(m, 2H, H5); 4, 63-4, 76(m, 1H, H4); 7, 18-7, 33(m, 5H arom.)
^{\mathring{1}}RMN ¹³C(50 MHz, CDCl₃): δ 26, 81(C3); 28, 34(C2); 40, 95(C5); 80, 56(C4); 126, 62(CH arom.); 128, 31(CH arom.); 129, 15(CH arom.); 135, 73(C6); 176, 93(Cl).
Key:
¹NMR
²Or
³And

| Microanalysis: | C % | H % |
|---|---|---|
| theoretical | 74.97 | 6.87 |
| actual | 74.85 | 7.00 |

^{\mathring{1}}SM(IE, 70 eV) m/e^{\acute{2}}(intensité relative %): 176(M⁺, 45), 91 [(M-lactone)⁺, 31], 85(lactone⁺, 100).
Key:
¹Mass spectrometry
²Relative intensity %

### 5) Process 5: Friedel-Crafts Reaction with Maleic Anhydride

### EXAMPLE

### Synthesis of p-(acetylamino)benzoyl-3 crotonic acid

2 g of acetanilide (0.015 mol) and 1.6 g of maleic anhydride (0.016 mol) are added to a suspension of AlCl₃ (7.5 g, 0.055 mol) in 1,2-dichloroethane (35 mL). The temperature climbs spontaneously to 60° C. The reaction medium is left to react for 20 h at room temperature and then is poured in ice and treated with concentrated HCl. The solid which precipitates is filtered and dried and then recrystallized in ethane 70% (melting point—m.p.—: 244° C.).

| | |
|---|---|
| MM(C₁₂H₁₁NO₄): 233.23 g Yield: 45% | 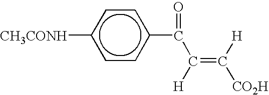 |

^{\mathring{1}}RMN ¹H(DMSO-d⁶): δ 7, 80(J_{AB} = Hz, 4Haryl); 6, 55(d, H_{A}, J_{AB} = 15 Hz); 7, 77(d, H_{B}, J_{AB} = 15 Hz); 2, 10(s, 3H, CH₃CONH).
Key:
¹NMR

### 6) Process 6: Reaction of a Methyl Ketone with Glyoxalic Acid

### EXAMPLE

### Synthesis of (E)-4-(1H-indol-3-yl)-4-oxobut-2-enoic acid

0.09 mL of ketone and 0.1 mol of glyoxylic acid are mixed and then brought to 140° C. for 20 h. A Hieckmann apparatus (displacement of water) is then mounted on the flask and heated to 110° C. for 10 h and 145° C. for 3 h. The crude reaction product is taken up with ethyl acetate and extracted with potassium bicarbonate. The aqueous phase is acidified with concentrated HCl at 0° C., and the precipitate obtained is filtered.

| | |
|---|---|
| Beige solid MM(C₁₂H₉NO₃): 215.21 g Yield: 62% T_{M} > 200° C. | 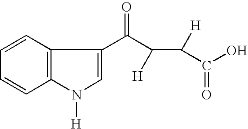 |

^{\mathring{1}}RMN(¹H 200 MHz,, CDCl₃): 8, 10-8, 30(m, 2H, CH indol. +NH); 6, 9-7, 7(m, 4H); 7, 80(d, 1H, CH_{A} = C, J³ = 16, 0 Hz); 6, 70(d, 1H, CH_{B} = C, J³ = 160 Hz).
Key:
¹NMR

### 7) Process 7: Reaction of Diethyl Succinate with Aryl Esters

### EXAMPLE

### a) Synthesis of diethyl 2-(pyridin-3-ylcarbonyl)succinate.

Ethyl nicotinate (10 mL, 73 mmol) is dissolved in ether (50 mL). Diethyl succinate (24 mL, 144 mmol) is added, followed by NaH (7.00 g, 60% in oil, 175 mmol) in small spatula loads. The reaction medium is stirred at room temperature in an argon atmosphere for 2 days. It is then poured over crushed ice.

The aqueous phase is collected and treated with 6N HCl in order to bring the pH to 4.5. The product is extracted with chloroform and dried using Na₂SO₄. After evaporation of the solvents, the crude oil obtained is purified by silica gel column chromatography, with AcOEt/Hexane 1/2 and then 1/1 and 7/3 as eluent.

| | |
|---|---|
| thick orange oil MM(C₁₄H₁₇NO₅): 279.29 g mass: 8.87 g Yield: 43% | 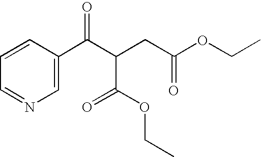 |

^{\mathring{1}}RMN(¹H, 300 MHz, CDCl₃): 9, 25(d, 1H, CHarom., J⁴ = 2, 0 Hz); 8, 81(dd, 1H, CHarom., J ³ = 4, 8 Hz et J⁴ = 2, 0 Hz); 8, 33-8, 29(M, 1H, CHarom.); 7, 47-7, 43(m, 1H, CHarom.); 4, 82(m, 1H, —CH—); 4, 18-4, 09(m, 4H, 2CH₂); 3, 24-2, 99(m, 2H, CH₂-COO-); 1, 28-1, 13(m, 6H, 2-CH₃).
Key:
¹NMR

### b) Synthesis of 4-oxo-4-pyridin-3-ylbutanoic acid

The keto ester synthesized in the preceding (8.87 g, 31.76 mmol) is dissolved in 1N sulfuric acid (80 mL). The reaction medium is stirred at reflux for 3 h and then cooled. The pH is brought to 4.5 by dropwise addition of a saturated solution of NaHCO₃. The precipitate which forms is filtered and dried.

| | |
|---|---|
| beige solid MM(C₉H₉NO₃): 179.18 g mass: 5.56 g Yield: 98% | 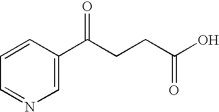 |

^{\mathring{1}}RMN(¹H, 300 MHz, DMSO): 9, 17(s, 1H, CHarom.); 8, 80(d, 1H, CHarom., J³ = 4, 3 H; 8, 32(d, 1H, CHarom., J³ = 7, 6 Hz); 7, 58(dd, 1H, CHarom., J³ = 4, 3 Hz et J³ = 7, 6 Hz); 3, 31(t, 2H, —CH₂—, J³ = 6, 2 Hz); 2, 62(t, 2H, —CH₂—, J³ = 6, 2 Hz).
Key:
¹NMR

### Process 8: Synthesis of a Compound of the Compound Type with General Formula VII:

### EXAMPLE

### Synthesis of 5(E)-[(4-chlorophenyl)methylidene]tetrahydrofuran-2-one (case in which R′═H)

In a dry 250-mL two-necked flask provided with a cooling device, in an argon atmosphere, one places tetrabutylammonium chloride (7.5 g, 1.5 Eq., 0.025 mol) which is dried under vacuum with a vane pump at 60° C overnight. 1-chloro-4-iodobenzene (4.07 g, 0.017 mol), 4-pentynoic acid (2.5 g, 1.5 Eq., 0.025 mol) and acetonitrile (50 mL) are added. Then with stirring, Pd(OAc)₂(PPh₃)₂ (5 mol %, 639 mg, 0.85 mmol) is added, followed by triethylamine (50 mL). The solution is heated to 60° C. for 1 h, cooled to room temperature and then poured quickly into 3N HCl. The aqueous phase is extracted with AcOEt (4×75 mL), and the organic phases are combined, washed with water (3×100 mL) then with brine (100 mL), dried using anhydrous sodium sulfate in the presence of activated charcoal, filtered using Celite and evaporated to dryness. Silica column chromatography gives the desired compound (2.18 g).

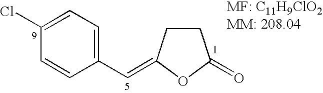

Chromatograph solvent: AcOEt/hexane, 20/80, Rf: 0.36 Yield: 61% Beige solid, MP = 140-141° C.

^{\mathring{1}}RMN ¹H(200 MHz, CDCl₃): δ 2, 69-2, 81(m, 2H, H2); 3, 07-3, 19(m, 2H, H3); 6, 24-6, 29(m, 1H, H5); 7, 23^{\mathring{2}}(systéme AB, 4H arom., J_{AB} = 8, 5 Hz, Δν 33 Hz).
^{\mathring{1}}RMN ¹³C(50 MHz, CDCl₃): δ 25, 05(C3); 27, 54(C2); 105, 96(C5); 128, 78(C7 ou C8); 128, 89(C7 ou C8); 132, 24(C6 ou C9); 132, 79(C6 ³ou C9); 151, 50(C4); 173, 92(C1).
Key:
1 NMR
2 System
3 Or

| Microanalysis: | C % | H % |
|---|---|---|
| theoretical | 63.32 | 4.35 |
| actual | 63.26 | 4.37 |

^{\mathring{1}}SM(IE, 70 eV) m/e^{\mathring{2}}(intensité relative %): 208 (M⁺, 94), 152[(M-CH₂CH₂CO)⁺, 72], 124[(H—C—Ph-pCl)⁺, 57], 89(100).
Key:
¹Mass spectrometry
²Relative intensity %

### B) Reduction of the Intermediate Keto Acids or Keto Esters with General Formula II Into Corresponding Alcohols III

### 1) Reduction of Keto Acids

### EXAMPLE

### Synthesis of 4-hydroxy-4-(1-tosyl-1H-pyrrol-3-yl)butanoic acid (Compound No. 4)

The keto acid (2.00 g, 62 mmol) is dissolved in a 1M aqueous solution of KHCO₃ (20 mL). KBH₄ (540 mg, 10 mmol) is added in small spatula loads. The reaction medium is stirred at room temperature for 12 h and then cooled to 0° C. Concentrated HCl is added dropwise to bring the pH to 1. It is left to be stirred cold for 1 h, and the precipitate which forms is then filtered and left to dry in the oven.

| | |
|---|---|
| white solid MM(C₁₅H₁₇NO₅S): 323.28 g mass: 1.80 g Yield: 89.5% | 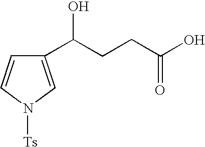 |

^{\mathring{1}}RMN(¹H, 300 MHz, DMSO): 7, 85(d, 2H, 2CHarom., J³ = 8, 1 Hz); 7, 44(d, 2H, 2CHarom. J³ = 8, 1 Hz); 7, 26(s, 1H, CHpyrrol.); 7, 12(s, 1H, CHpyrrol.); 6, 32(s, 1H, CHpyrrol.); 4, 42(t, 1H, CH—O—, J³ = 6, 2 Hz); 2, 39(s, 3H, —CH₃); 2, 20 (m, 2H, —CH₂—); 1, 77(m, 2H, —CH₂—).
Key:
¹NMR

### 2) Reduction of Keto Esters with General Formula II by Switching to Lactones with General Formula VI

### EXAMPLE

### Synthesis of 5-(1H-indol-5-yl)dihydrofuran-2(3H)-one

The keto ester (300 mg, 1.10 mmol) is dissolved in dry MeOH (10 mL). NaBH4 (46 mg, 1.22 mmol) is added. The reaction medium is stirred at room temperature for 24 h, with addition every 6 h of a spatula tip of NaBH₄. The methanol is evaporated, and the residue is taken up in a water/AcOEt mixture. The organic phase is collected, dried using Na₂SO₄ and evaporated.

The crude product is purified by silica gel column chromatography with CH₂Cl₂/AcOEt 9/1 as eluent.

| | |
|---|---|
| white powder MM(C₁₂H₁₁NO₂): 201.23 g mass: 110 g Yield: 47% | 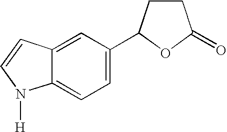 |

^{\mathring{1}}RMN(¹H, 300 MHZ, CDCl₃): 8, 58\acute{2}(large s, 1H, NH); 7, 61(s, 1H, CHarom.); 7, 38(d, 1H, CHarom., J³ = 9, 3 Hz); 7, 23(m, 1H, CHindol.); 7, 13(d, 1H, CHarom., J³ = 9, 3 Hz); 6, 55-6, 54(m, 1H, CHindol.); 5, 58(m, 1H, CHlacton); 2, 76-2, 57(m, 3H, CH₂—CH); 2, 38-2, 22(m, 1H, CH).
Key:
¹NMR
²Broad

The acid alcohols can be lactonized by hydrolysis with heat (60° C., concentrated HCl in THF) for one night.

### C) Access to the Sodium Salts (Final Products)

The acid alcohols III obtained by reduction followed by hydrolysis or opening of the lactones IV are salified by an alkaline solution, for example, by a 1M solution of sodium carbonate in water (0.9 Eq.). The aqueous phase is washed with ethyl acetate, then lyophilized or evaporated, followed by trituration with ether.

This salification reaction is well-known to the expert in the field and does not need to be developed in more detail here. Of course, the expert in the field will be able to choose, instead of the aforementioned sodium hydroxide, any other base suitable for obtaining the desired corresponding salts. As examples of suitable bases, it is possible to mention, in a nonlimiting manner, mineral bases such as LiOH, Ca(OH)₂, etc., as well as organic bases conventionally used in synthesis organic chemistry, in particular those used for the synthesis of compositions intended for pharmaceutical use.

### D) Particular Cases:

### 1) Formation of Amides with General Formula V (cf. FIG. 2)

As indicated in the diagram of FIG. 2, the opening of reduced lactones IV can be done by addition of a primary or secondary amine or by addition of a hydroxamic acid, in order to end up with the amides or with the corresponding hydroxamino-substituted V. Three embodiments given as nonlimiting examples relating to this operation will hereafter be given in more detail.

### Example 1

### Synthesis of a primary amide, 4-(1-benzothiophen-2-yl)-4-hydroxybutanamide (amide derivative of Compound No. 18)

The lactone (103 mg, 0.47 mmol) is dissolved in THF (5 mL), and NH₄0H (2 mL, 25% in water) is added. It is heated to 50° C. for 1 h 30 min and left to cool and evaporate the THF. The residue is taken up in AcOEt and dried using Na₂SO₄. The solvents are evaporated, and the crude oil which is obtained is purified by silica gel column chromatography, with CH₂Cl₂/MeOH 9/1 as eluent.

| | |
|---|---|
| Thick oil MM(C₁₂H₁₃NO₂S): 235.31 g mass: 61 g Yield: 55% | 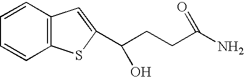 |

^{\mathring{1}}RMN(1H, 300 MHz, CDCl₃): 7, 89-7, 85(m, 2H, 2CHarom.); 7, 44(s, 1H, CHthienyl); 7, 41-7, 33(m, 2H, 2CHarom.) 5, 59-et 5, 45(2s, 2H, —NH₂); 5, 24(m, 1H, CH—O); 3, 50(s, 1H, —OH); 2, 50-2, 21(m, 4H, —CH₂—CH₂).
Key:
¹NMR

### Example 2

### Synthesis of a secondary amide, 4(1-benzothiophen-2-yl)-N-benzyl-4-hydroxybutanamide

The lactone (0.102 g, 0.47 mmol) is dissolved in ether (5 mL). Benzylamine (51 μL, 0.47 mmol) is added, and the reaction medium left with stirring for 48 h. The ether is evaporated, and the crude product is purified by silica gel column chromatography, with CH₂Cl₂/MeOH 95/5 as eluent.

| | |
|---|---|
| very thick translucent oil MM(C₁₉H₂₀NO₂S): 326.45 g mass: 107 g Yield: 70% | 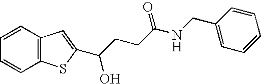 |

^{\mathring{1}}RMN(¹H, 300 MHz, CDCl₃): 7, 84(m, 2H, 2CHarom.); 7, 43-7, 28(m, 8H, 8CHarom.); 5, 89(large s, 1H, —OH); 5, 24(m, 1H, CH—O); 4, 46(d, 2H, CH2, J³ = 5, 0 Hz); 3, 82(d, 1H, —NH, J³ = 5, 0 Hz); 2, 47-2, 19(m, 4H, —CH₂—CH₂—).
Key:
¹NMR
²Broad

### Example 3

### Treatment with Hydroxamic Acid in Order to Obtain N,4-dihydroxy-4-phenylbutanamide (Compound No. 34)

Metallic sodium (100 mg, 4.34 mmol) is dissolved in methanol (4 mL). Hydroxylamine (0.30 g, 4.32 mmol) is added, and then lactone (0.50 g, 3.09 mmol) dissolved in methanol (5 mL). The reaction medium is stirred at room temperature for 12 h. The methanol is evaporated, and the residue is taken up in ethyl acetate. The insoluble material is filtered, and the filtrate is dried using Na₂SO₄. The solvents are evaporated, and the oil which is obtained is triturated with ether.

| | |
|---|---|
| very hygroscopic white solid MM(C₁₀H₁₃NO₃): 195.22 g mass: 220 mg Yield: 37% | 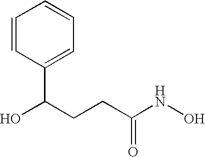 |

^{\mathring{1}}RMN(¹H, 300 MHz, DMSO): 7, 41-7, 21(m, 5H, 5CHarom.); 4, 55(m, 1H, —CH—O); 2, 04-1, 73(2m, 4H, —CH₂CH₂—).
Key:
¹NMR

### 2) Syntheses of Compounds of Types VIII, IX and X

As shown in the diagram of FIG. 3, the presence in position 3 or 4 of an amine on ethyl β-benzoyl propionate (compound with general formula II with Ar=PhNH₂, n=0, X═(CH₂)₂ and R=Et) made possible the synthesis of dihydroquinolones (compounds VIII) and phenylquinoxalines (compounds X).

In effect, condensation of the amine with Meldrum acid leads, as intermediate, to the amine methylene-dioxane-dione (compound IX) which is cyclized thermally (cf. left diagram of FIG. 3).

The reaction with phenylglyoxal leads to the expected phenylquinoxaline with formula X.

The keto esters which are obtained are then reduced to lactones and salified as described in the diagram of FIG. 1.

The following indicative examples describe in more detail the conditions of operation used for these types of reactions.

### a) Synthesis of Compounds of Type VIII

### Example 1

### Synthesis of ethyl 4-(4-{[2,2-dimethyl-4,6-dioxo-1,3-dioxan-5-ylidene)methyl]amino}phenyl)-4-oxobutanoate

The mixture, Meldrum acid (0.70 g, 0.46 mmol) and methyl orthoformate (15 mL), is heated to reflux (140° C.) for 2 h. The amine (1.00 g, 4.52 mmol) is added all at once. The reaction mixture is stirred at reflux for 12 h. After cooling, the solvent is evaporated, and the residue is purified by recrystallization in methanol.

| | |
|---|---|
| orange-gold powder MM(C₁₉H₂₁NO₇): 375.39 g mass: 1.26 g Yield: 75% TM: 154° C. | 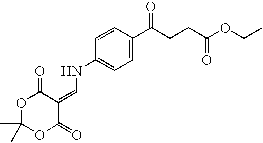 |

^{\mathring{1}}RMN(¹H, 300 MHz, CDCl₃): 11, 32(large d, 1H, —NH, J³ = 14, 0 Hz); 8, 70(d, 1H, CH, J³ = 14, 0 Hz); 8, 07(m, 2H, 2CHarom.); 7, 34(m, 2H, 2CHarom.); 4, 15(q, 2H, —CH₂—, J³ 7, 2 Hz); 3, 29(t, 2H, −CH2—, J³ = 6, 5 Hz); 2, 77(t, 2H, —CH₂—, J³ 6, 5 Hz); 176 (s, 6H, 2-CH3); 1, 27(t, 3H, —CH3, J³ = 7, 2 Hz).
Key:
¹NMR
²Broad

### Example 2

### Synthesis of ethyl 4-oxo-4-(4-oxo-1,4-dihydroquinolin-6-yl) butanoate

Diphenyl ether (10 mL) is heated to 240° C. (slight reflux); the Meldrum derivative (1.00 g, 2.66 mmol) is added all at once. The reaction mixture is then stirred at 240° C. for 10 min and then poured very slowly over petroleum ether (150 mL). The precipitate which forms is filtered and purified by silica gel column chromatography (eluent: CH₂Cl₂/MeOH 9/1).

| beige solid MM (C₁₅H₁₅NO₄): 273.33 g mass: 0.52 g Yield: 71% | 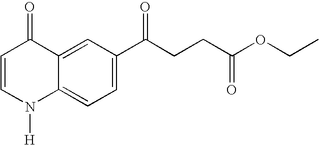 |
|---|---|
| ^{\mathring{1}}RMN (¹H, 200 MHz, CDCl₃ + MeOD): 8.90(d, 1H, CHarom., J⁴=2.0 Hz); 8.13(dd, 1H, CHarom., J³=8.8 Hz, J⁴=2.0 Hz); 7.62(d, 1H, CHvinyl., J³=7.3 Hz); 7.37(d, 1H, CHarom., J³=8.8 Hz); 6.30(d, 1H, CHvinyl., J³=7.3 Hz); 4.16(q, 2H, —CH₂—, J³=7.1 Hz); 3.41(t, 2H, —CH₂—, J³=6.6 Hz); 2.77(t, 2H, —CH₂—, J³=6.6 Hz); 1.26(t, 3H, —CH₃, | |
| #J³=7.1 Hz). | |

Key: 1 NMR

### b) Syntheses of Compounds of Type X

### Example 1

### 4-[4-(acetylamino)-3-nitrophenyl]-4-oxobutanoic acid

Fuming HNO₃ (12 mL) is cooled to 0° C., and the derivative which is to be nitrated (4.00 g, 17.0 mmol) is then quickly added. It is left to be stirred at 0° C. for 10 min. Then, the medium is poured over ice, and the precipitate which forms is filtered.

| | |
|---|---|
| yellow powder MM (C₁₂H₁₂N₂O₆): 280.23 g mass: 2.97 g Yield: 62% T_{M}: 145° C. | 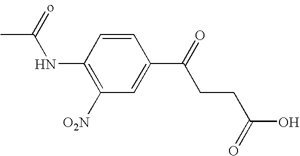 |

^{\mathring{1}}RMN (¹H, 200 MHz, DMSO): 10.6(s, 1H, NH); 8.44(d, 1H, CHarom., J⁴=2.0 Hz); 8.29(dd, 1H, CHarom., J³=8.5 Hz, J⁴=2.0 Hz); 7.86(d, 1H, CHarom., J³=8.5 Hz), 3.30(t, 2H, —CH₂—, J³=6.2 Hz); 2.61 (t, 2H, —HC₂—, J³=6.2 Hz).
Key: 1 NMR

### Example 2

### Ethyl 4-(4-amino-3-nitrophenyl)-4-oxobutanoate

The N-acetylated derivative (2.00 g, 7.51 mmol) is mixed with concentrated HCl (10 mL) and heated to reflux for 15 min. Then, EtOH (50 mL) is added, and it is stirred at reflux for 12 h. It is then allowed to cool, and the solvents are evaporated to the maximum extent. The residue is extracted with ethyl acetate, and the pH is brought to 8-9 by addition of KHCO₃. The organic phase is dried, and the solvents are evaporated. Finally, the crude product is purified by silica gel column chromatography (eluent: AcOEt/Hexane 1/3).

| | |
|---|---|
| orange oil MM (C₁₂H₁₄N₂O₅): 266.25 g mass: 1.36 g Yield: 68% | 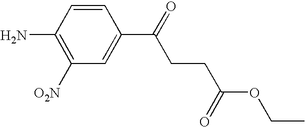 |

^{\mathring{1}}RMN (¹H, 300 MHz, CDCl₃): 8.81(d, 1H, CHarom., J⁴=2.0 Hz); 8.02(dd, 1H, CHarom., J³=8.8 Hz, J⁴=2.0 Hz); 6.86(d, 1H, CHarom., J³=8.8 Hz); 4.17(q, 2H, —CH2-, J³=7.1 Hz); 3.26(t, 2H, —CH₂—, J³=6.5 Hz); 2.77(t, 2H, —CH₂—, J³=6.5 Hz); 1.27(t, 3H, —CH₃, J³=7.1 Hz).
Key: 1 NMR

### Example 3

### Ethyl 4-(3,4-diaminophenyl)-4-oxobutanoate

The nitrated derivative (1.31 g, 4.9 mmol) is dissolved in 95% ethanol (30 mL). SnCl₂, 2H₂O (4.4 g, 19.6 mmol) is added and heated to reflux for 2 h. The solvents are evaporated, and the residue is extracted with ethyl acetate; then, the pH is brought to 7 by addition of KHCO₃. The precipitate of tin salts which forms is filtered, and the organic phase is dried using Na₂SO₄ and evaporated to dryness. Finally, the purification of the crude product is done by silica gel column chromatography (eluent: AcOEt/Hexane 1/1).

| | |
|---|---|
| Thick oil MM (C₁₂H₁₆N₂O₃): 236.01 g mass: 700 mg Yield: 60% | 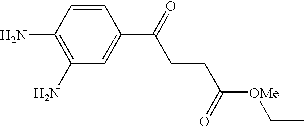 |

^{\mathring{1}}RMN (¹H, 300 MHz, CDCl₃): 7.46-7.38(m, 2H, 2CHarom.,); 6.68(d, 1H, CHarom., J³=8.1 Hz); 4.16(q, 2H, —OCH₂—); 3.89(large^{\mathring{2}}s, 2H, —NH₂); 33.37(large^{\mathring{2}}s, 2H, —NH₂); 3.22(t, 2H, —CH₂—, J³=6.8 Hz); 2.72(t, 2H, —HC₂—, J³=6.8 Hz); 1.27(t, 3H, —CH₃).
Key:
1 NMR
2 Broad

### Example 4

### Ethyl-4-oxo-4-(2-phenylquinoxalin-6-yl) butanoate

In a round-bottomed flask, the diamino ester is dissolved in EtOH (10 mL). Phenylglyoxal, hydrate (130 mg, 0.85 mmol) is added and heated to reflux for 5 h. It is allowed to cool, and the precipitate which forms is filtered.

| | |
|---|---|
| yellow powder MM (C₂₀H₁₈N₂O₃): 334.37 g mass: 233 mg Yield: 82% | 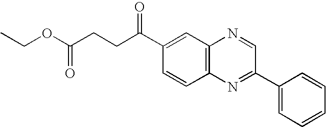 |

^{\mathring{1}}RMN (¹H, 200 MHz, CDCl₃): 9.43(s, 2H, CHquinox.); 8.74(d, 1H, CHarom., J⁴=1.9 Hz); 8.36(dd, 1H, CHarom., J³=8.9 Hz, J⁴=1.9 Hz); 8.26-8.19(m, 4H, 4CHarom.,; 7.60-7.58(m, 3H, 3CHarom.); 4.19(q, 2H, —CH₂—, J³=7.1 Hz); 3.50(t, 2H, —CH₂—, J³=6.6 Hz); 2.86(t, 2H, —CH₂—, J³=6.6 Hz); 1.30(t, 3H, —CH₃, J³=7.1 Hz).
Key: 1 NMR

### E) Syntheses of Enantiomerically Pure Compounds

The present invention also describes the enantioselective synthesis of the (R) and (S) acid sodium salts of γ-benzyl-γ-hydroxybutanoic acid. These syntheses involve the L or D glutamic acid as illustrated in the diagram of FIG. 4. For this, optically pure (R) and (S) acid chlorides were prepared according to processes described in the literature (ref. 1: M. Larcheveque et al., Bull. Soc. Chim. Fr. 1987,116-122; G. Eguchi et al., Bull. Chim. Soc. Jpn., 1974, 47, 1704-1708).

These compounds were then treated with tetraphenyltin in the presence of a catalyst in HMPT (ref. 2: J. W. Labadie et al., J.A.C.S., 1983, 105, 6129). The (R) and (S) γ-benzoyl-γ-butyrolactones are then obtained, which are reduced by catalytic hydrogenation (process 4), giving the two optically pure enantiomers (compounds VI′ (R) and VI″ (S). These are salified as described in the preceding, leading to the corresponding (R) and (S) sodium salts.

### F) Characterization of the Compounds Which are Obtained

The following table gives the main physicochemical characteristics of specific compounds belonging to the families with general formula I, I′ or I″ as synthesized in practice and which are explicitly claimed in the present invention. Of course, the corresponding acid compounds with general formula I or I′ in which W is COOH, which enable one to obtain the sodium salts, are also considered to be part of this table and of the explicitly claimed compounds.

TABLE 2
Main physicochemical characteristics of the compounds which are obtained

| | | | | E | | | |
|---|---|---|---|---|---|---|---|
| A | | C | D | Masse | F | | |
| N° du | B | N° du | formule | molaire | RMN “¹H”, 200 ou 300MHz, solvant | | |
| composé | Nom | Procédé | brute | g/mol | ou microanalyses | | |
| 1 | Sel de sodium de l'acide | 1 | C₁₀H₁₀O₃Na | 201.18 | Microanalyses | | |
| | 4-hydroxy-4- | | | | | C % | H % |
| | phénylbutanoïque | | | | Th. | 59.70 | 5.01 |
| | | | | | G Pr. | 59.41 | 5.14 |
| 2 | Sel de sodium de l'acide | 1 | C₁₀H₉Cl₂O₃Na | 271.08 | ¹H, 200MHz, D₂O: 7.35-7.30(m, 2H, | | |
| | 4-(3,4 dichlorophényl)- | | | | 2CHarom.); 7.11-7.06(m, 1H, CHarom.); | | |
| | 4-hydroxybutanoïque | | | | 4.50(t, 1H, CH—O—, J³=6.6Hz); 2.08-2.00(m, | | |
| | | | | | 2H, —CH₂—); 1.88-1.75(m, 2H, —CH₂—) | | |
| 3 | Sel de sodium de l'acide | 1 | C₁₂H₁₅O₅Na | 262.23 | ¹H, 200MHz, D₂O: 6.97(m, 3H, | | |
| | 4-(3,4- | | | | CHarom.); 4.59(t, 1H, CH—O—, | | |
| | diméthoxylphényl)-4- | | | | J³=8.3Hz); 3.82(s, 3H, —O—CH₃—); 3.80(s, | | |
| | hydroxybutanoïque | | | | 3H, —O—CH₃—); 2.16-1.89(m, 4H, —CH₂—CH₂—) | | |
| 4 | Sel de sodium de l'acide | 1 | C₁₅H₁₆NO₅SNa | 345.37 | ¹H, 300MHz, D₂O: 7.63(d, 2H, 2CHarom., | | |
| | 4-hydroxy-4-(1-tosyl- | | | | J³=7.7Hz); 7.22(d, 2H, 2CHarom., | | |
| | 1H-pyrrol-3-yl)- | | | | J³=7.7Hz); 7.13(s, 1H, CHpyrrol.); | | |
| | butanoïque | | | | 6.29(s, 2H, 2 CHpyrrol.); 4.47(t, 1H, | | |
| | | | | | CH—O—, J³=6.4Hz); 2.22(s, 3H, —CH₃—); | | |
| | | | | | 2.03-1.82(2m, 4H, —CH₂—CH₂) | | |
| 5 | Sel de sodium de l'acide | 7 | C₉H₁₀O₃Na | 189.17 | ¹H, 200MHz, D₂O: 8.36 J (large s, 2H, | | |
| | 4-hydroxy-4-pyridin-4- | | | | 2CHarom.); 7.33 J (large s, 2H, 2CHarom.); | | |
| | yl-butanoïque | | | | 4.75(m, 1H CH—O); 2.13-1.89(m, 4H, —CH₂—CH₂) | | |
| 6 | Sel de sodium de l'acide | 7 | C₉H₁₀O₃Na | 189.17 | ¹H, 200MHz, D₂O: 8.34(m, 2H, 2CHarom.); | | |
| | 4-hydroxy-4-pyridin-3- | | | | 7.74(d, 1H, CHarom., J³=8.0Hz); 7.33(dd, | | |
| | yl-butanoïque | | | | 1H, CHarom., J³=4.9 M et | | |
| | | | | | 8.0Hz); 4.65(t, 1H, CH—O—, J³=6.3Hz); | | |
| | | | | | 2.15-1.89(m, 4H, —CH₂—CH₂) | | |
| 7 | Sel de sodium de l'acide | 1 | C₈H₉NaO₃S | 208.22 | ¹H, 300MHz, D₂O: 7.34-7.32(m, 1H, | | |
| | 4-(2-thiényl)-4- | | | | CHthinyl.); 7.01-6.69(m, 2H, | | |
| | hydroxybutanoïque | | | | 2CHthinyl.); 4.91(t, 1H, CH—O—, | | |
| | | | | | J³=6.8Hz); 2.26-1.99(m, 4H, —CH₂—CH₂) | | |
| 8 | Sel de sodium de l'acide | 1 | C₁₂H₁₄NO₄Na | 259.24 | ¹H, 200MHz, D₂O: 7.24(m, 4H, | | |
| | 4-[4- | | | | 4CHarom.); 4.51(t, 1H, J³=6.8Hz, CH—O); | | |
| | acétylamino)phényl]-4- | | | | 1.84-2.07(m, 4H —CH₂—CH₂); 1.93(s, 3H, —CH₃) | | |
| | hydroxybutanoïque | | | | | | |
| 9 | Sel de sodium de l'acide | 1 | C₁₁H₁₄NO₅Na | 263.23 | ¹H, 300MHz, D₂O: 7.00(s, 1H, | | |
| | 4-[5-(éthoxycarbonyl)-1H-pyrrol-3- | | | | CHpyrrol.); 6.90(s, 1H, CHpyrrol.); 4.58(t, | | |
| | yl]-4-hydroxybutanoïque | | | | 1H, CH—O—, J³=6.2Hz); 4.20(q, 2H, —CH₂—, | | |
| | | | | | J³=7.2Hz); 2.14-1.92(2m, 4H, —CH₂—CH₂); | | |
| | | | | | 1.25(t, 3H, —CH₃, J³=7.2Hz) | | |
| 10 | Sel de sodium de l'acide | 1 | C₁₂H₁₃O₄Na | 244.22 | ¹H, 300MHz, D₂O: 7.17(s, 1H, CHarom.); | | |
| | 4-(2,3-dihydro-1- | | | | 7.01(d, 1H, CHarom., J³=8.1Hz); 6.69(d, | | |
| | benzofuran-5-yl)-4- | | | | 1H, CHarom., J³=8.1Hz); 4.47(m, 3H, —CH₂ | | |
| | hydroxybutanoïque | | | | et CH—O); 3.09(t, 2H, —CH₂—, | | |
| | | | | | J³=8.7Hz); 2.08-1.78(m, 4H, —CH₂—CH₂) | | |
| 11 | Sel de sodium de l'acide | 1 | C₁₂H₁₃O₅Na | 260.92 | ¹H, 300MHz, D₂O: 6.82 J (large s, 3H, | | |
| | 4-(2,3-dihydro-1,4- | | | | 3CHarom.); 4.48(t, 1H, —CH—0—, | | |
| | benzodioxin-6-yl)-4- | | | | J³=8.2Hz); 4.18(s, 4H, O—CH2—CH2—O); | | |
| | hydroxybutanoïque | | | | 2.15-1.78(m, 4H, —CH₂—CH₂—) | | |
| 12 | Sel de sodium de l'acide | 1 | C₁₄H₁₆NO₄Na | 285.28 | ¹H, 200MHz, D₂O): 7.82(d, 1H, CHarom., | | |
| | 4-(1-acétyl-2,3-dihydro- | | | | J³=8.6Hz); 7.16(s, 1H, CHarom.); 7.08(d, | | |
| | 1H-indol-5-yl)-4- | | | | 1H, CHarom., J³=8.6Hz); 4.52(t, 1H, —CH—O, | | |
| | hydroxybutanoïque | | | | J³=6.4Hz); 3.93(t, 2H, —CH₂, | | |
| | | | | | J³=8.2Hz); 3.02(t, 2H, —CH₂, J³=8.2Hz); | | |
| | | | | | 2.07(s, 3H, —CH₃); 2.04-1.88(2m, 4H, | | |
| | | | | | —CH₂—CH₂—) | | |
| 13 | Sel de sodium de l'acide | 1 | C₁₄H₁₃O₃Na | 252.14 | ¹H, 300MHz, D₂O: 8.10(d, 1H, | | |
| | 4-hydroxy-4-(1- | | | | CHarom.); 7.89-7.78(m, 2H, 2CHarom.); | | |
| | naphthyl)-butanoïque | | | | 7.58-7.44(m, 4H, 4CHarom.); 5.44(t, 1H, —CH—O, | | |
| | | | | | J³=5.8Hz); 2.24-1.98(m, 4H, —CH₂—CH₂—) | | |
| 14 | Sel de sodium de l'acide | 1 | C₁₄H₁₃O₃Na | 252.14 | ¹H, 300MHz, D₂O: 7.84-7.82(m, 3H, | | |
| | 4-hydroxy-4-(2- | | | | 3CHarom.); 7.75(s, 1H, CHarom.); 7.48-7.42(m, | | |
| | naphthyl)-butanoïque | | | | 3H, 3CHarom.); 2.15-1.94(m, 4H, | | |
| | | | | | —CH₂—CH₂—) | | |
| 15 | Sel de sodium de l'acide | 1 | C₁₃H₁₅O₃Na | 242.25 | ¹H, 300MHz, D₂O: 7.17(s, 1H, CHarom.); | | |
| | 4-(2,3-dihydro-1H- | | | | 7.15(s, 1H, CHarom.); 7.15(d, 1H, | | |
| | inden-5-yl)-4-hydroxy- | | | | CHarom., J³=7.6Hz); 7.05(d, 1H, | | |
| | butanoïque | | | | CHarom., J³=7.6Hz); 4.54(t, 1H, CH—O, | | |
| | | | | | J³=6.5Hz); 2.80-2.73(m, 4H, 2—CH₂); | | |
| | | | | | 2.14-1.82(m, 3H, 3—CH₂) | | |
| 16 | Sel de sodium de l'acide | 1 | C₁₅H₁₆NO₅Na | 313.28 | ¹H, 200MHz, D₂O: 7.56(s, 1H, CHarom.); | | |
| | 4-[2-(éthoxycarbonyl)- | | | | 7.32-7.36(m, 2H, 2CHarom.); 7.05(s, 1H, | | |
| | 1H-indol-5-yl]-4- | | | | CHindol.); 4.71(m, 1H, —CH—O); 4.20(q, | | |
| | hydroxybutanoïque | | | | 2H, —CH₂—); 2.20-2.10(m, 4H, | | |
| | | | | | —CH₂—CH₂—); 1.3(t, 3H, —CH₃—) | | |
| 17 | Sel de sodium de l'acide | 1 | C₁₂H₁₂NO₃Na | 241.22 | ¹H, 200MHz, D₂O: 7.55(s, 1H, CHarom.); | | |
| | 4-hydroxy-4-(1H-indol- | | | | 7.43(d, 1H, CHarom., J³=8.4Hz); 7.30(d, | | |
| | 5-yl)-butanoïque | | | | 1H, CHindol., J³=2.9Hz); 7.13(d, 1H, | | |
| | | | | | CHarom., J³=8.4Hz); 6.48(d, 1H, | | |
| | | | | | CHindol., J³=2.9Hz); 4.70 K (en dessous du | | |
| | | | | | pic de l'eau, 1H, CH—O); 2.17-1.91(m, | | |
| | | | | | 4H, —CH₂—CH₂—) | | |
| 18 | Sel de sodium de l'acide | 1 | C₁₂H₁₁SONa | 258.28 | ¹H, 300MHz, D₂O: 7.89-7.84(m, 2H, | | |
| | 4-(1-benzothiophène-2- | | | | 2CHarom.); 7.44(s, 1H, CHthio.); 7.40-7.30(m, | | |
| | yl)-4-hydroxybutanoïque | | | | 2H, 2CHarom.); 5.01(t, 1H, —CH—O, | | |
| | | | | | J³=4.0Hz); 2.15(m, 4H, —CH₂—CH₂—) | | |
| 19 | Sel de sodium de l'acide | H | C₁₃H₁₂NO₄Na | 269.23 | ¹H, 300MHz, D₂O: 8.08(d, 1H, CHarom., | | |
| | 4-hydroxy-4-(4-oxo- | cas | | | J³=8.1Hz); 8.00(d, 1H, CHarom., J³=7.2Hz); | | |
| | 1,4-dihydroquinolin-7- | parti- | | | 7.54(s, 1H, CHarom.); 7.41(d, | | |
| | yl)-butanoïque | culier | | | 1H, CHarom., J³=8.1Hz); 6.37(d, 1H, | | |
| | | | | | CHarom., J³=7.2Hz); 4.80(t, —CH—O, | | |
| | | | | | J³=6.4Hz); 2.15(t, 2H, —CH₂, J³=7.2Hz); | | |
| | | | | | 1.99(t, 2H, —CH₂, J³=7.2Hz) | | |
| 20 | Sel de sodium de l'acide | H | C₁₃H₁₂NO₄Na | 269.23 | ¹H, 300MHz, D₂O: 7.93(d, 1H, CHarom., | | |
| | 4-hydroxy-4-(4-oxo- | cas | | | J⁴=1.9Hz); 7.86(d, 1H, CHvinyl., J³=7.2Hz); | | |
| | 1,4-dihydroquinolin-6- | parti- | | | 7.61(dd, 1H, CHarom., J³=8.8Hz, | | |
| | yl)-butanoïque | culier | | | J⁴=1.9Hz); 7.45(d, 1H, CHarom., | | |
| | | | | | J³=8.8Hz); 6.27(d, 1H, CHvinyl., J³=7.2Hz); | | |
| | | | | | 4.75(t, 1H, —CH—O, J³=3.7Hz); | | |
| | | | | | 2.15(t, 2H, —CH₂, J³=7.2Hz); | | |
| | | | | | 2.17-1.94(2m, 4H, —CH₂—CH₂—) | | |
| 21 | Sel de sodium de l'acide | 2 | C₁₂H₁₃N₂O₃Na | 256.23 | ¹H, 200MHz, D₂O: 8.08(d, 1H, CHarom., | | |
| | 4-hydroxy-4-(7-méthyl- | | | | J³=6.6Hz); 7.37(s, 1H, CHimidaz.); 7.19(s, | | |
| | imidazo[1,2-a]pyridin- | | | | 1H, CHarom.); 6.72(d, 1H, CHarom., | | |
| | 3-yl)-butanoïque | | | | J³=6.6Hz); 4.93(m, 1H, —CH—O); 2.21(s, | | |
| | | | | | 3H, —CH₃); 2.16-2.27(m, 4H, —CH₂—CH₂—) | | |
| 22 | Sel de sodium de l'acide | 1 | C₁₂H₁₂NO₄Na | 257.28 | ¹H, 200MHz, D₂O: 12.31(s, 1H, NH); | | |
| | 4-hydroxy-4-(2-oxo- | | | | 7.16(s, 1H, CHarom.); 7.08(d, 1H, | | |
| | 2,3-dihydro-1H-indol-5- | | | | CHarom., J³=8.8Hz); 6.74(d, 1H, | | |
| | yl)-butanoïque | | | | CHarom., J³=8.8Hz); 4.47(s, 1H, CHO); | | |
| | | | | | 3.44(s, 2H, CH₂); 2.21 et 1.79(2s, 4H, 2CH₂) | | |
| 23 | Sel de sodium de l'acide | 1 | C₁₅H₁₇N₂O₄Na | 312.30 | ¹H, 300MHz, D₂O): 7.60(s, 1H, CHarom.); | | |
| | 4-{2- | | | | 7.28-,7.42(m, 2H, 2CHarom.); 7.05(s, | | |
| | [(diméthylamino)carbon | | | | 1H, CHindol.); 3.35(m, 4H, —CH₂—CH₂—); | | |
| | yl]-1H-indol-5-yl}-4- | | | | 2.50(s, 6H, 2—CH₃) | | |
| | hydroxybutanoïque | | | | | | |
| 24 | Sel de sodium de l'acide | 1 | C₁₆H₁₅O₃Na | 278.29 | ¹H, 200MHz, D₂O: 7.68(d, 1H, CHarom., | | |
| | 4-(1,2- | | | | J³=8.6Hz); 7.46-7.35(m, 2H, 2CHarom.); | | |
| | dihydroacénaphthylen-4-yl)-4- | | | | 7.20(m, 2H, 2CHarom.); 5.23(t, 1H, | | |
| | hydroxybutanoïque | | | | —CH—O, J³=6.1Hz); 3.17(t, 4H, —CH₂—CH₂—, | | |
| | | | | | J³=7.6Hz); 2.16-2.00(m, 4H, —CH₂—CH₂—) | | |
| 25 | Sel de sodium de l'acide | 1 | C₁₆H₁₃O₃SNa | 308.34 | ¹H, 200MHz, D₂O: 8.10-8.00(m, 2H, | | |
| | 4-dibenzo[b,d]thiophène- | | | | 2CHarom.); 7.98-7.75(m, 2H, | | |
| | 2-yl-4- | | | | 2CHarom.); 7.40-7.30(m, 3H, | | |
| | hydroxybutanoïque | | | | 3CHarom.); 4.74(m, 1H, —CH—O); | | |
| | | | | | 2.15-2.02(m, 4H, —CH₂—CH₂—) | | |
| 26 | Sel de sodium de l'acide | 1 | C₁₆H₁₃O₄Na | 292.27 | ¹H, 300MHz, DMSO: 8.04(m, 2H, | | |
| | 4-dibenzo[b,d]furan-2- | | | | 2CHarom.); 7.70-7.35(m, 5H, 5CHarom.); | | |
| | yl-4-hydroxybutanoïque | | | | 4.78(m, 1H, —CH—O); 2.13(2H, | | |
| | | | | | —CH₂); 1.84(m, 2H, —CH₂—) | | |
| 27 | Sel de sodium de l'acide | 1 | C₁₈H₁₆O₄NNa | 333.33 | ¹H, 300MHz, D₂O: 7.57-7.01(m, 7H, | | |
| | 4-(9-acétyl-9H- | | | | 7CHarom.); 4.64(m, 1H, —CH—O); 2.30(s, | | |
| | carbazol-3-yl-4- | | | | 3H, —CH₃); 2.20-1.82(2m, 4H, —CH₂—CH₂—) | | |
| | hydroxybutanoïque | | | | | | |
| 28 | Sel de sodium de l'acide | 1 | C₁₆H₁₃O₄SNa | 324.34 | ¹H, 200MHz, D₂O: 7.06-6.83(m, 7H, | | |
| | 4-hydroxy-4- | | | | 7CHarom.); 4.43(m, 1H, —CH—O); | | |
| | (phénoxathiin-2 ou 3- | | | | 2.01-1.77(2m, 4H, —CH₂—CH₂—) | | |
| | yl)butanoïque | | | | | | |
| | isomère A | | | | | | |
| 29 | Sel de sodium de l'acide | 1 | C₁₆H₁₃O₄SNa | 324.34 | ¹H, 300MHz, D₂O: 7.77-7.63(m, 4H, | | |
| | 4-hydroxy-4- | | | | 4CHarom.); 738-7.32(m, 3H, 3CHarom.); | | |
| | (phénoxathiin-2 ou 3- | | | | 4.62(m, 1H, —CHO); 2.23-1.96(2m, 4H, | | |
| | yl)butanoïque | | | | —CH₂—CH₂—) | | |
| | isomère B | | | | | | |
| 30 | Sel de sodium de | | C₂₀H₂₀NO₄Na | 361.38 | ¹H, 200MHz, D₂O: 7.32-7.16(m, 7H, | | |
| | l'acide 4-(5-acétyl- | | | | 7CHarom.); 7.51(m, 1H, —CH—O—); | | |
| | 10,11-dihydro-5H- | | | | 3.27-3.07(m, 2H, —CH₂—); 2.78-2.64(m, | | |
| | dibenzo[b,f]azépin-3- | | | | 2H, —CH2—); 1.97-1.76(m, 7H, | | |
| | yl)-4-hydroxybutanoïque | | | | —CH₂—CH₂— et —CH₃) | | |
| 31 | Sel de sodium de l'acide | 1 | C₁₇H₁₅O₄Na | 306.30 | ¹H, 200MHz, D₂O: 7.16-6.91(m, 7H, | | |
| | 4-hydroxy-4-(9H- | | | | 7CHarom.); 4.58(m, 1H, —CH—O); 3.86(s, | | |
| | xanthen-2- | | | | 2H, —CH₂); 2.11-1.97(2m, 4H, —CH₂—CH₂) | | |
| | yl)butanoïque | | | | | | |
| 32 | Sel de sodium de l'acide | 1 | C₁₇H₁₅O₃Na | 290.30 | ¹H, 300MHz, D₂O: 7.65(m, 2H, | | |
| | 4-(9H-fluoren-3- | | | | 2CHarom.); 7.45(m, 2H, 2CHarom.); | | |
| | yl)hydroxybutanoïque | | | | 7.29-7.23(m, 3H, 3CHarom.); 4.62(m, 1H, | | |
| | | | | | —CH—O); 3.37(s, 2H, —CH2—); | | |
| | | | | | 2.17-1.91(2m, 4H, —CH2—CH2—) | | |
| 33 | Sels de sodium des | 1 | C₁₄H₁₂N₃O₃Na | 293.25 | ¹H, 300MHz, D₂O: 8.06(s, 1H, CHarom. | | |
| | acides 4-hydroxy-4-(4- | | | | L des 2 isomères); 7.50(s, 1H, CHarom. L des 2 | | |
| | oxo-4,5-dihydro-3H- | | | | isomères); 7.39(d, 1H, CHarom. iso. 6, | | |
| | pyridazo(4,5-b)indol-8 | | | | J³=7.6Hz); j 7.28(2d, 1H, CHarom. iso. 8 | | |
| | et 6-yl)butanoïque | | | | M et CHarom. iso. 6, J³=8.7Hz); 7.16(d, 1H, | | |
| | mélange isomère 8/6 | | | | CHarom. iso. 8, J³=8.7Hz); 7.06(t, 1H, | | |
| | | | | | CHarom. iso. 6, J³=7.6Hz); 4.90(m, 1H, | | |
| | | | | | CH—O— iso. 6); 4.66(m, 1H, CH—O— iso. 8); | | |
| | | | | | 2.29-1.95(2m, 4H, —CH₂—CH₂— L des 2 isomères) | | |
| 34 | N,4-dihydroxy-4- | H | C₁₀H₁₃NO₃ | 195.22 | ¹H, 300MHz, DMSO: 7.41-7.21(m, 5H, | | |
| | phénylbutanamide | cas | | | 5CHarom.); 4.55(m, 1H, —CH—O); | | |
| | | parti- | | | 2.04-1.73(2m, 4H, —CH₂—CH₂—) | | |
| | | culier | | | | | |
| 35 | Sel de sodium de l'acide | 4 | C₁₁H₁₃NaO₃ | 216.21 | Microanalyses: | | |
| | 4-hydroxy-5- | | | | | C % | H % |
| | phénylpentanoïque. | | | | Th. | 56.40 | 6.45 |
| | | | | | G Pr. | 56.94 | 6.15 |
| 36 | Sel de sodium de l'acide | I | C₁₁H₁₃NaO₃ | 216.21 | Microanalyses: | | |
| | 4(R)-hydroxy-5- | énantio- | | | | C % | H % |
| | phénylpentanoïque. | mère | | | Th. | 56.40 | 6.45 |
| | | pur | | | G Pr. | 56.50 | 6.39 |
| 37 | Sel de sodium de l'acide | I | C₁₁H₁₃NaO₃ | 216.21 | Microanalyses: | | |
| | 4(S)-hydroxy-5- | énantio- | | | | C % | H % |
| | phénylpentanoïque. | mère | | | Th. | 56.40 | 6.45 |
| | | pur | | | G Pr. | 56.47 | 6.44 |
| 38 | Sel de sodium de l'acide | 8 | C₁₁H₁₁Cl₂NaO₃ | 285.10 | Microanalyses: | | |
| | 5-(3,4-dichlorophényl)- | | | | | C % | H % |
| | 4-hydroxypentanoïque. | | | | Th. | 46.34 | 3.89 |
| | | | | | G Pr. | 46.12 | 3.90 |
| 39 | Sel de sodium de l'acide | 3 | C₁₁H₁₃O₃Na | 216.21 | ¹H, 200MHz, D₂O: 7.20(m, 5H, | | |
| | 5-hydroxy-5- | | | | 5CHarom.); 4.49(t, 1H, CH—O); 2.04(t, | | |
| | phénylpentanoïque | | | | 2H, CH₂COO, J³=6.5Hz); 1.36-1.63(m, | | |
| | | | | | 4H, CH₂—CH₂) | | |
| 40 | Sel de sodium de l'acide | 5 | C₁₅H₁₆NO₄Na | 297.26 | ¹H, 200MHz, D₂O: 7.30(s, 1H, CHarom.); | | |
| | 4-(3,3-diméthyl-2-oxo- | | | | 7.13(d, 1H, CHarom., J³=7.9Hz); 6.84(d, | | |
| | 1,2,3,4- | | | | 1H, CHarom., J³=7.9Hz); 6.55(m, 1H, | | |
| | tétrahydroquinolin-6- | | | | CH═C; 5.95(d, 1H, CH═C, J³=10.8Hz); | | |
| | yl)-4-hydroxybutanoïque | | | | 5.24(d, 1H, CH—O, J³=3.3Hz); 2.35(s, | | |
| | | | | | 2H, CH₂); 1.17(s, 6H, 2CH₃) | | |
| 41 | Sel de sodium de l'acide | 6 | C₁₀H₈O₃CINa | 234.60 | Microanalyses: | | |
| | (E)-4-(4-chlorophényl)- | | | | | C % | H % |
| | 4-hydroxybut-2-ènoïque | | | | Th. | 56.48 | 4.27 |
| | | | | | Pr. | 56.37 | 4.02 |

| | | | | | |
|---|---|---|---|---|---|
| 42 | Sel de sodium de l'acide | 6 | C₁₂H₁₂NO₄Na | 257.23 | ¹H, 200MHz, D₂O: 720(m, 4H, |
| | (E)-4-(4- | | | | CHarom.); 6.56(dd, 1H, J_{AB}=15.6Hz; |
| | acétylaminophényl)-4- | | | | J_{AX}=5.6Hz); 5.94(d, 1H, J_{AB}=15.6Hz); |
| | hydroxybut-2-ènoïque | | | | 5.16(d, J_{AX}=5.6Hz, CH—O); 1.95(s, 3H, CH₃CO) |

Key:
A Compound No.
B Name
C Process No.
D Total formula
E Molar mass g/mol
F “¹H” NMR, 200 or 300 MHz, solvent or microanalyses
G Actual
H Particular case
I Pure enantiomer
J Broad
K Above the peak of water
L Of the 2 isomers
M And
1 Sodium salt of 4-hydroxy-4-phenylbutanoic acid
2 Sodium salt of 4-(3,4 dichlorophenyl)-4-hydroxybutanoic acid
3 Sodium salt of 4-(3,4-dimethoxyphenyl)-4-hydroxybutanoic acid
4 Sodium salt of 4-hydroxy-4-(1-tosyl-1H-pyrrol-3-yl)butanoic acid
5 Sodium salt of 4-hydroxy-4-pyridin-4-ylbutanoic acid
6 Sodium salt of 4-hydroxy-4-pyridin-3-ylbutanoic acid
7 Sodium salt of 4-(2-thienyl)-4-hydroxybutanoic acid
8 Sodium salt of 4-[4-(acetylamino)phenyl]-4-hydroxybutanoic acid
9 Sodium salt of 4-[5-(ethoxycarbonyl)-1H-pyrrol-3-yl]-4-hydroxybutanoic acid
10 Sodium salt of 4-(2,3-dihydro-1-benzofuran-5-yl)-4-hydroxybutanoic acid
11 Sodium salt of 4-(2,3-dihydro-1,4-benzodioxin-6-yl)-4-hydroxybutanoic acid
12 Sodium salt of 4-(1-acetyl-2,3-dihydro-1H-indol-5-yl)-4-hydroxybutanoic acid
13 Sodium salt of 4-hydroxy-4-(1-naphthyl)butanoic acid
14 Sodium salt of 4-hydroxy-4-(2-naphthyl)butanoic acid
15 Sodium salt of 4-(2,3-dihydro-1H-inden-5-yl)-4-hydroxybutanoic acid
16 Sodium salt of 4-[2-(ethoxycarbonyl)-1H-indol-5-yl]-4-hydroxybutanoic acid
17 Sodium salt of 4-hydroxy-4-(1H-indol-5-yl)butanoic acid
18 Sodium salt of 4-(1-benzothiphen-2-yl)-4-hydroxybutanoic acid
19 Sodium salt of 4-hydroxy-4-(4-oxo-1,4-dihydroquinolin-7-yl)butanoic acid
20 Sodium salt of 4-hydroxy-4-(4-oxo-1,4-dihydroquinolin-6-yl)butanoic acid
21 Sodium salt of 4-hydroxy-4-(7-methylimidazo[1,2-a]pyridin-3-yl)butanoic acid
22 Sodium salt of 4-hydroxy-4-(2-oxo-2,3-dihydro-1H-indol-5-yl)butanoic acid
23 Sodium salt of 4-{2-[(dimethylamino)carbonyl]-1H-indol-5-yl}-4-hydroxybutanoic acid
24 Sodium salt of 4-(1,2-dihydroacenaphthylen-4-yl)-4-hydroxybutanoic acid
25 Sodium salt of 4-dibenzo[b,d]thiophen-2-yl-4-hydroxybutanoic acid
26 Sodium salt of 4-dibenzo[b,d]furan-2-yl-4-hydroxybutanoic acid
27 Sodium salt of 4-(9-acetyl-9H-carbazol-3-yl-4-hydroxybutanoic acid
28 Sodium salt of 4-hydroxy-4-(phenoxathiin-2 or 3-yl)butanoic acid isomer A
29 Sodium salt of 4-hydroxy-4-(phenoxathiin-2 or 3-yl) butanoic acid isomer B
30 Sodium salt of 4-(5-acetyl-10,11-dihydro-5H-dibenzo[b,f]azepin-3-yl)-4-hydroxybutanoic acid
31 Sodium salt of 4-hydroxy-4-(9H-xanthen-2-yl)butanoic acid
32 Sodium salt of 4-(9H-fluoren-3-yl)hydroxybutanoic acid
33 Sodium salts of 4-hydroxy-4-(4-oxo-4,5-dihydro-3H-pyridazo(4,5-b)indol-8 and 6-yl)butanoic acids 8/6 isomer mixture
34 N,4-dihydroxy-4-phenylbutanamide
35 Sodium salt of 4-hydroxy-5-phenylpentanoic acid
36 Sodium salt of 4(R)-hydroxy-5-phenylpentanoic acid
37 Sodium salt of 4(S)-hydroxy-5-phenylpentanoic acid
38 Sodium salt of 5-(3,4-dichlorophenyl)-4-hydroxypentanoic acid
39 Sodium salt of 5-hydroxy-5-phenylpentanoic acid
40 Sodium salt of 4-(3,3-dimethyl-2-oxo-1,2,3,4-tetrahydroquinolin-6-yl)-4-hydroxybutanoic acid
41 Sodium salt of (E)-4-(4-chlorophenyl)-4-hydroxybut-2-enoic acid
42 Sodium salt of (E)-4-(4-acetylaminophenyl)-4-hydroxybut-2-enoic acid

### G) Tests of Therapeutic Activity

### 1) In Vitro Tests

Approximately forty compounds according to the invention were synthesized and tested in experiments of binding on the brains of rats using ³H-GHB as radioligand and according to the experimental protocol described hereafter.

For the displacement studies, the reference ligand used is tritiated GHB (100 Ci/mmol, CEA, Saclay). The receptors studied come from membranes of brains of Wistar rats raised in the laboratory. The animals are sacrificed quickly by decapitation, and the brains are removed excluding the cerebellum and the brain stem.

The brains are then homogenized in 10 volumes (weight/volume) of 0.32M sucrose containing 5 mM EDTA (pH 6.0). After a first centrifugation at 800 G intended for eliminating the cellular debris and the nuclei, the supernatant is centrifuged at 16,000 G so as to eliminate the P₂ residue (synaptosomes+mitochondria).

This residue is then homogenized in a suitable apparatus, for example, of the type known by the term “polytron,” in 70 volumes of distilled water at 0° C. containing 5 mM EDTA. After centrifugation at 20,000 G (4° C., 20 min), the residue obtained is washed with the same medium supplemented with 0.5% CHAPS (3-[3-cholamidopropyldimethylammonio]-1-propane sulfonate). After another centrifugation at 20,000 G, the membranes obtained are resuspended in a potassium phosphate buffer with pH=6.0. After recentrifugation, the residue is stored at −80° C. for use the next day.

The incubation of the membranes with the radioactive GHB and the various compounds at variable concentrations (between 10⁻⁹ and 10⁻⁴ M) is done in a potassium phosphate buffer also with pH=6.0 for a duration of 30 min in ice.

After this incubation, the separation of the bound ³H-GHB from the free ³H-GHB is done by filtration under suction with filters made of glass fiber (Whatmann GF/B). The membranes retained by the filter are quickly washed under suction three times with 1 mL of incubation buffer maintained at 0° C.

The filters are then “counted” by liquid scintillation in a counter, in the presence of a scintillation liquid. The results are expressed in percentage of total reversible binding determined in the presence of an excess of nonradioactive GHB (500 μM, 60-80% total binding). The statistical analyses of the IC₅₀ values (concentration of synthetic analogues capable of displacing 50% of the reversible binding of the ³H-GHB) are summarized in the table hereafter. The lower the IC₅₀ value, the greater the affinity of the ligand for the receptor. The statistical analyses of the displacement curves are carried out using the GraphPad Prism software (San Diego, Calif.).

The results obtained are gathered in the following table:

TABLE 3
In vitro results

| \mathring{1} | |
|---|---|
| N° du | \mathring{2} |
| composé | CI₅₀ μM |
| GHB | 5.60 |
| 1 | 6.80 |
| 2 | 0.30 |
| 3 | 0.30 |
| 4 | 1.60 |
| 5 | 3.90 |
| 6 | 2.50 |
| 7 | 1.80 |
| 8 | 0.80 |
| 9 | 2.30 |
| 10 | 0.60 |
| 11 | 0.90 |
| 12 | 0.60 |
| 13 | 0.10 |
| 14 | 0.30 |
| 15 | 0.70 |
| 16 | 0.20 |
| 17 | 1.40 |
| 18 | 0.10 |
| 19 | 1.10 |
| 20 | 24.7 |
| 21 | 16.2 |
| 22 | 1.20 |
| 23 | 0.20 |
| 24 | 0.08 |
| 25 | 0.1 |
| 26 | 0.30 |
| 27 | 0.20 |
| 28 | 0.10 |
| 29 | 0.90 |
| 30 | 0.50 |
| 31 | 0.10 |
| 32 | 0.30 |
| 33 | 0.20 |
| 34 | 23.5 |
| 35 | 2.30 |
| 36 | 1.80 |
| 37 | 25.0 |
| 38 | 0.80 |
| 39 | 34.2 |
| 40 | 1.10 |
| 41 | 3.80 |
| 42 | 0.60 |

Key:
\mathring{1} Compound No.
\mathring{2} IC₅₀ μM

As can be observed, certain compounds among those synthesized are approximately ten times more active than GHB and therefore have improved sedative properties.

The compounds of the present invention are therefore of particular value with regard to their use for obtaining a drug intended for the treatment of neurological or mental disorders in which the central nervous system plays a part. This pertains in particular to disorders in which the GHB receptors are involved and which can benefit from the effects of an agonist or an antagonist of the GHB receptors: regulation of sleep and secretion of hormones, in particular of growth hormones, reduction of anxiety or increased alertness, antiepileptic activity, regulation of weight and food intake, regulation of mood or antidepressive activity, neuroleptic activity, regulation of circadian rhythm, hypnotic or anesthetic activity, neuroprotective or anti-ischemic activity, activity in the process of drug withdrawal and in addiction.

These drugs are characterized by the fact that they contain, as active ingredient, at least one compound with general formula I, I′ or I″.

Preferabley, the aforementioned compound(s) used as active ingredient is(are) one or more sodium salts with general formula I″ obtained by neutralization of a compound with general formula I or I′ containing an acid function for the group W.

### 2) In Vivo Trials: Electroencephalographic Study (EEG) of Rats Who Received 2-4 mmol/kg of GHB Synthesis

Analogues

Male Wistar rats, weighing 200 g at the beginning of the experiment, coming from the Centre d'Elevage Janvier (Route des Chenes-Secs, Le Genest St-Isle, 53940 France) were used for this study.

After eight days of adaptation to the breeding conditions at the Animalerie Centrale de la Faculte de Medecine (11, rue Humann 67084 Strasbourg), these animals were placed in individual cages (Makrolon type cages 3H, 425×266×150) in a standard (7 a.m./7 p.m.) day/night rhythm, with water and food (UAR ref. A04) continually available to them. The animals were then transferred to an experimentation facility on supports capable of receiving 24 cages.

### a) Implantation of Frontoparietal Cortical Electrodes

The surgical procedure is the following: after one month of becoming accustomed to the (10 a.m./10 p.m.) day/night cycle, the rats are weighed and then anesthetized with ketamine (Imalgene 500 Merial) at a dose of 150 mg/kg i.p. After having placed the animal in a stereotactic frame (Narishige), a rostrocaudal incision is made using a sterilized scalpel (No. 3 blade, Swann-Morton England).

The parietal bone sutures, Lambda and Bregma, are exposed serve as stereotactic reference (point 0). After having perforated the cranial casing using a dental drill without infringing on the meninges (Minitor Narishige), two stainless steel screws 500 μm in diameter (Magister, 4 rue du Lac 25130 Villers Le Lac) are implanted at the following coordinates: Bregma AP: ±4 mm and ML: ±3 mm.

Two copper wires are soldered with tin to the screws, on one hand, and to the female connector, on the other hand (VP Electronic, Square de la Poteme, 91302 Massy Cedex). The assembly which is realized is then covered with a polymerizing resin (Paladur, Kulzer, Germany).

The implanted rats are then placed in their respective cages. A post-operative period greater than 48 h is complied with before any recording.

### b) EEG Recording

All the EEG recordings are made during the first hours of the dark [sic; light] phase (that is, between 10 a.m. and 1 p.m.), which represents the period of awakening and intense activity of the animals.

The rats are placed in a cage made of Plexiglas (170×170×300), and after a 30 min period of becoming accustomed to their new environment, they are recorded continuously for a duration of 3 h after i.p. injection (2 mL/kg) of 0.9% NaCl or the ligand to be studied. The EEG graph is made using an 8-track recorder (Alvar Electronic, 6 rue du Progres, 93511-Montreuil) with a running speed of 0.5 cm/s.

### c) Calculation of the Duration of Slow Wave Sleep (SWS)

The total durations of slow wave sleep for each animal are evaluated in 20 min sections for the total duration of the 3 h recording (1 cm=2 seconds).

### d) Statistical Analysis and Graphic Representation

The statistical comparison is made using an analysis of variance test (Anova) followed by a multiple comparison test. The animals (6 to 8/group/dose) are recorded both on 0.9% NaCl (reference value) and after administration of the product to be studied in the amount of 2 mmol/kg.

The results are represented by expressing the means ±SEM of the total durations in min of slow wave sleep in 20 min time sections with respect to the affinity of the ligand for the GHB receptor (IC₅₀) in the table hereafter. The numbers in parentheses correspond to the percentages of slow wave sleep with respect to the total duration of sleep.

TABLE 4
In vivo results

| | | | | Augmentation |
|---|---|---|---|---|
| N° du | | Dosage | Durée de | de la durée du |
| Composé | CI₅₀ | utilisé | sommeil lent | sommeil lent |
| testé | μM | mmole/kg i.p. | profond (mn) | profond (mn) |
| \mathring{1} | \mathring{2} | \mathring{3} | \mathring{4} | \mathring{5} |
| NaCl | — | — | 11 ± 4 (7%) | — |
| 2 | 0.30 | 0.28 | 53 ± 7 | 42 (23%) |
| 24 | 0.08 | 0.28 | 61 ± 8 | 50 (28%) |
| 24 | 0.08 | 0.15 | 53 ± 7 | 42 (23%) |

Key:
\mathring{1} No. of the tested compound
\mathring{2} IC₅₀ μM
\mathring{3} Dosage used mmol/kg i.p.
\mathring{4} Duration of slow wave sleep (min)
\mathring{5} Increase of the duration of slow wave sleep (min)

As can be observed, the compounds according to the invention make possible a significant increase of the duration of slow wave sleep.

Consequently, the present invention also relates to a pharmaceutical composition containing, as active ingredient, at least one compound with general formula I, I′ or a salt with general formula I″.

The claimed pharmaceutical compositions moreover contain other pharmaceutically acceptable excipients or vehicles.

The present invention makes possible the use of a compound according to said invention for obtaining a drug containing, as active ingredient, at least one compound with general formula I, I′ or I″ for the treatment of a disease which can be treated by administration of an agonist or antagonist of GHB receptors, in particular, neurological or mental disorders of the central nervous system and, in particular, regulation of sleep and of secretion of hormones, in particular, growth hormones, reduction of anxiety or increased alertness, antiepileptic activity, regulation of weight and food intake, regulation of mood or antidepressive activity, neuroleptic activity, regulation of circadian rhythm, hypnotic or anesthetic activity, neuroprotective or anti-ischemic activity, activity in the process of drug withdrawal and in addiction.

Thanks to the compounds of the present invention, it also becomes possible to propose a process for treatment of a disease in a mammal which can be treated by administration of a GHB agonist, in particular, diseases of the central nervous system, and particularly, diseases relating to sleep and to anxiety, said treatment including the administration to the mammal of a therapeutically effective quantity of at least one compound with general formula I, I′ or I″ according to the present invention, preferably at least one sodium salt chosen from the compounds with general formula I″, in particular those mentioned in Table 2.

Of course, the invention is not limited to the embodiments which have been described. Modifications remain possible, particularly from the standpoint of the constitution of the various elements or by substitution of equivalent techniques, without consequently leaving the field of protection of the invention.

1. Compounds with general formula I 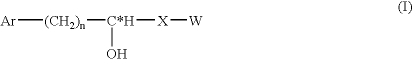
in which Ar represents one of the following mono-, bi- or tricyclics: 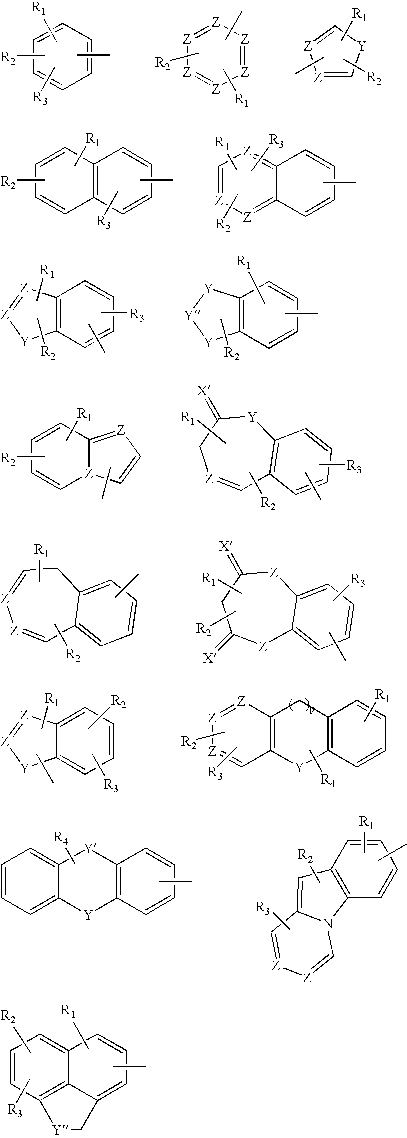
in which:
R₁, R₂, R₃ and R independently represent a hydrogen atom, a halogen, an alkyl group, an aryl group, an aralkyl group, a hydroxyl group, a methoxy group, an acetyl group, a tosyl group, a COOEt group, an NHCOCH₃ group, an NH2 group, a CON(CH₃)₂ group, an NO₂ group or a COR₅R₆ group, in which R₅ and R₆ independently represent a hydrogen atom, a methyl group, a CH₃H₇ group or a benzyl group,
each z independently represents a nitrogen or carbon atom,
Y and Y′ independently represent a carbon, sulfur, oxygen or nitrogen atom,
Y″ represents a methylene, ethylene or propylene group,
each X′ independently represents a sulfur or oxygen atom,
p has a value of 0, 1 or 2,
in which n has a value of 0 or 1,
in which X independently represents (CH₂)₂ or (CH₂)₃ or X═—CH═CH— (trans) and in which W represents COOH, COO⁻M⁺(M⁺ representing a counterion which is pharmaceutically acceptable), CH₂OH, COOR (with R representing an alkyl group), SO₃H or PO₃H₂ or a group chosen from the following: 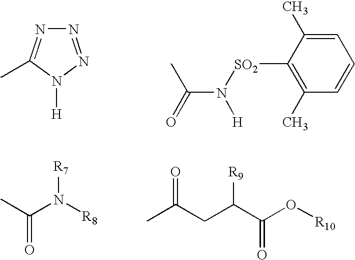
in which R₇ and R₈ independently represent a hydrogen atom, an alkyl group, an aryl group, an aralkyl group or a hydroxyl group,
in which R₉ independently represents a hydrogen atom or a methyl group and in which R₁₀ independently represents an ethyl, C₁₂H₁₅ or adamantyl group.

2. Compounds according to claim 1 with general formula I′: 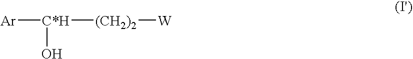
in which W represents COOH or COO⁻M⁺ (M⁺ representing a counterion which is pharmaceutically acceptable), and in which Ar represents the groups defined in claim 1.

3. A compound according to claim 1 or 2, characterized by the fact that it pertains to the salts with general formula I″ 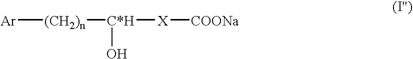
in which Ar, X and n are defined in claim 1 or 2.

4. A compound according to claim 3, characterized by the fact that it pertains to the sodium salt of 4-hydroxy-4-phenylbutanoic acid.

5. A compound according to claim 3, characterized by the fact that it pertains to the sodium salt of 4-(3,4-dichlorophenyl)-4-hydroxybutanoic acid.

6. A compound according to claim 3, characterized by the fact that it pertains to the sodium salt of 4-(3,4-dimethoxyphenyl)-4-hydroxybutanoic acid.

7. A compound according to claim 3, characterized by the fact that it pertains to the sodium salt of 4-hydroxy-4-(1-tosyl-1H-pyrrol-3-yl)butanoic acid.

8. A compound according to claim 3, characterized by the fact that it pertains to the sodium salt of 4-hydroxy-4-pyridin-4-ylbutanoic acid.

9. A compound according to claim 3, characterized by the fact that it pertains to the sodium salt of 4-hydroxy-4-pyridin-3-ylbutanoic acid.

10. A compound according to claim 3, characterized by the fact that it pertains to the sodium salt of 4-(2-thienyl)-4-hydroxybutanoic acid.

11. A compound according to claim 3, characterized by the fact that it pertains to the sodium salt of 4-[4-(acetylamino)phenyl]-4-hydroxybutanoic acid.

12. A compound according to claim 3, characterized by the fact that it pertains to the sodium salt of 4-[5-(ethoxycarbonyl)-1H-pyrrol-3-yl]-4-hydroxybutanoic acid.

13. A compound according to claim 3, characterized by the fact that it pertains to the sodium salt of 4-(2,3-dihydro-1-benzofuran-5-yl)-4-hydroxybutanoic acid.

14. A compound according to claim 3, characterized by the fact that it pertains to the sodium salt of 4-(2,3-dihydro-1,4-benzodioxin-6-yl)-4-hydroxybutanoic acid.

15. A compound according to claim 3, characterized by the fact that it pertains to the sodium salt of 4-(1-acetyl-2,3-dihydro-1H-indol-5-yl)-4-hydroxybutanoic acid.

16. A compound according to claim 3, characterized by the fact that it pertains to the sodium salt of 4-hydroxy-4-(1-naphthyl)butanoic acid.

17. A compound according to claim 3, characterized by the fact that it pertains to the sodium salt of 4-hydroxy-4-(2-naphthyl)butanoic acid.

18. A compound according to claim 3, characterized by the fact that it pertains to the sodium salt of 4-(2,3-dihydro-1H-inden-5-yl)-4-hydroxybutanoic acid.

19. A compound according to claim 3, characterized by the fact that it pertains to the sodium salt of 4-[2-(ethoxycarbonyl)-1H-indol-5-yl]-4-hydroxybutanoic acid.

20. A compound according to claim 3, characterized by the fact that it pertains to the sodium salt of 4-hydroxy-4-(1H-indol-5-yl)butanoic acid.

21. A compound according to claim 3, characterized by the fact that it pertains to the sodium salt of 4-(1-benzothiphen-2-yl)-4-hydroxybutanoic acid.

22. A compound according to claim 3, characterized by the fact that it pertains to the sodium salt of 4-hydroxy-4-(4-oxo-1,4-dihydroquinolin-7-yl)butanoic acid.

23. A compound according to claim 3, characterized by the fact that it pertains to the sodium salt of 4-hydroxy-4-(4-oxo-1,4-dihydroquinolin-6-yl)butanoic acid.

24. A compound according to claim 3, characterized by the fact that it pertains to the sodium salt of 4-hydroxy-4-(7-methylimidazo[1,2-a]pyridin-3-yl)butanoic acid.

25. A compound according to claim 3, characterized by the fact that it pertains to the sodium salt of 4-hydroxy-4-(2-oxo-2,3-dihydro-1H-indol-5-yl)butanoic acid.

26. A compound according to claim 3, characterized by the fact that it pertains to the sodium salt of 4-{2-[(dimethylamino)carbonyl]-1H-indol-5-yl}-4-hydroxybutanoic acid.

27. A compound according to claim 3, characterized by the fact that it pertains to the sodium salt of 4-(1,2-dihydroacenaphthylen-4-yl)-4-hydroxybutanoic acid.

28. A compound according to claim 3, characterized by the fact that it pertains to the sodium salt of 4-dibenzo[b,d]thiophen-2-yl-4-hydroxybutanoic acid.

29. A compound according to claim 3, characterized by the fact that it pertains to the sodium salt of 4-dibenzo[b,d]furan-2-yl-4-hydroxybutanoic acid.

30. A compound according to claim 3, characterized by the fact that it pertains to the sodium salt of 4-(9-acetyl-9H-carbazol-3-yl-4-hydroxybutanoic acid.

31. A compound according to claim 3, characterized by the fact that it pertains to the sodium salt of 4-hydroxy-4-(phenoxathiin-2-yl)butanoic acid.

32. A compound according to claim 3, characterized by the fact that it pertains to the sodium salt of 4-hydroxy-4-(phenoxathiin-3-yl)butanoic acid.

33. A compound according to claim 3, characterized by the fact that it pertains to the sodium salt of 4-(5-acetyl-10,11-dihydro-5H-dibenzo[b,f]azepin-3-yl)-4-hydroxybutanoic acid.

34. A compound according to claim 3, characterized by the fact that it pertains to the sodium salt of 4-hydroxy-4-(9H-xanthen-2-yl)butanoic acid.

35. A compound according to claim 3, characterized by the fact that it pertains to the sodium salt of 4-(9H-fluoren-3-yl)hydroxybutanoic acid.

36. A compound according to claim 3, characterized by the fact that it pertains to the sodium salt of 4-hydroxy-4-(4-oxo-4,5-dihydro-3H-pyridazo(4,5-b)indol-8-yl)butanoic acid.

37. A compound according to claim 3, characterized by the fact that it pertains to the sodium salt of 4-hydroxy-4-(4-oxo-4,5-dihydro-3H-pyridazo(4,5-b)indol-6-yl)butanoic acid.

38. A compound according to claim 1, characterized by the fact that it pertains to N,4-dihydroxy-4-phenylbutanamide.

39. A compound according to claim 1, characterized by the fact that it pertains to the sodium salt of 4-hydroxy-5-phenylpentanoic acid.

40. A compound according to claim 1, characterized by the fact that it pertains to the sodium salt of 4(R)-hydroxy-5-phenylpentanoic acid.

41. A compound according to claim 1, characterized by the fact that it pertains to the sodium salt of 4(S)-hydroxy-5-phenylpentanoic acid.

42. A compound according to claim 1, characterized by the fact that it pertains to the sodium salt of 5-(3,4-dichlorophenyl)-4-hydroxypentanoic acid.

43. A compound according to claim 1, characterized by the fact that it pertains to the sodium salt of 5-hydroxy-5-phenylpentanoic acid.

44. A compound according to claim 3, characterized by the fact that it pertains to the sodium salt of 4-(3,3-dimethyl-2-oxo-1,2,3,4-tetrahydroquinolin-6-yl)-4-hydroxybutanoic acid.

45. A compound according to claim 1, characterized by the fact that it pertains to the sodium salt of (E)-4-(4-chlorophenyl)-4-hydroxybut-2-enoic acid.

46. A compound according to claim 1, characterized by the fact that it pertains to the sodium salt of (E)-4-(4-acetylaminophenyl)-4-hydroxybut-2-enoic acid.

47. Use of a compound according to any one of claims 1 to 46 for obtaining a drug containing, as active ingredient, at least one compound with general formula I, I′ or I″ according to any one of claims 1 to 46 for the treatment of a disease which can be treated by administration of an agonist or antagonist of GHB receptors, in particular, neurological or mental disorders of the central nervous system and, in particular, regulation of sleep and of secretion of hormones, in particular, growth hormones, reduction of anxiety or increasing of alertness, antiepileptic activity, regulation of weight and food intake, regulation of mood or antidepressive activity, neuroleptic activity, regulation of circadian rhythm, hypnotic or anesthetic activity, neuroprotective or anti-ischemic activity, activity in the process of drug withdrawal and in addiction.

48. Use according to claim 47, characterized by the fact that the compound(s) used as active ingredient is(are) one or more sodium salts with general formula I″ obtained by neutralization of a compound with general formula I or I′ according to claim 1 or 2 and containing an acid function for the group W.

49. A pharmaceutical composition characterized by the fact that it contains, as active ingredient, at least one compound with general formula I, I′ or I″ according to any one of claims 1 to 46.
